(19) United States
(12) Patent Application Publication
(10) Pub. No.: US 2022/0259287 A1
(43) Pub. Date: Aug. 18, 2022

(54) SINGLE CHAIN FACTOR VIII MOLECULE

(71) Applicant: Biotest AG, Dreieich (DE)

(72) Inventors: Steffen Kistner, Frankfurt am Main (DE); Christopher Ungerer, Langen (DE); Jens Daufenbach, Mainz (DE); Peter Herbener, Kirchhain (DE)

(21) Appl. No.: 17/438,646

(22) PCT Filed: May 8, 2020
(86) PCT No.: PCT/EP2020/062818

§ 371 Date: Sep. 13, 2021

(30) Foreign Application Priority Data
May 9, 2019 (EP) 19173440.9

(51) Int. Cl. C07K 14/755; A61P 7/04

(52) U.S. Cl. CPC C07K 14/755; A61K 38/00; A61P 7/04

(57) ABSTRACT

The present invention relates to a recombinant Factor VIII protein comprising, in a single chain, a heavy chain portion comprising an A1 and an A2 domain and a light chain portion comprising an A3, C1 and C2 domain of Factor VIII, wherein the B-domain is partially deleted in two deletions, the first leading to the presence of a defined processing sequence cleavable by thrombin, and the second leading to absence of the furin cleavage recognition site at position R1664-R1667. An internal fragment of the B-domain is maintained Nucleic acids encoding said protein, host cells and methods of preparing the protein are also provided, as well as a pharmaceutical composition comprising the protein, nucleic acid or host cell, which may be used for treatment of hemophilia A.

The present invention relates to a recombinant Factor VIII (FVIII) protein comprising, in a single chain, a heavy chain portion comprising an A1 and an A2 domain and a light chain portion comprising an A3, C1 and C2 domain of Factor VIII, wherein the B-domain is partially deleted in two deletions, the first leading to the presence of a defined processing sequence cleavable by thrombin, and the second leading to absence of the furin cleavage recognition site. An internal fragment of the B-domain is maintained. Nucleic acids encoding said protein, host cells and methods of preparing the protein are also provided, as well as a pharmaceutical composition comprising the protein, nucleic acid or host cell, which may be used for treatment of hemophilia A.

FVIII is an important co-factor in the coagulation cascade. Natural FVIII is synthesized as a single chain protein and is afterwards cleaved by intracellular proteases. The secreted FVIII is a heterodimer with a truncated B domain. If the coagulation cascade is activated, FVIII is cleaved by thrombin at three positions, leading to a heterotrimer and loss of the B domain (heterotrimeric FVllla). The heterotrimer builds a complex with the activated coagulation Factor IXa and coagulation Factor X, thereby promoting the activation of the latter one.

The human natural FVIII single chain protein consists of 2351 amino acids. The first 19 amino acids comprise the signal sequence which is cleaved prior to secretion, leading to a FVIII molecule comprising 2332 amino acids. Additionally, cleavage occurs at the C-terminal part of the B domain, leading to a mature heterodimeric FVIII. The heavy chain of the heterodimer comprises the domains A1 (residues 20-355), A2 (392-729) and B (760-1667), whereas the light chain comprises the domains A3 (1709-2038), C1 (2039-2191) and C2 (2192-2351). Moreover, there are three spacer regions: a1 (356-391), a2 (730-759) and a3 (1668-1708).

Annotation of amino acids in the FVIII molecule differs between authors. This is due to the 19 amino acid signal sequence which can be included into the amino acid count or can be omitted (the above annotation is based on inclusion of the signal sequence). This variation of plus or minus 19 amino acids is a frequent difference in numeration for full-length FVIII sequences in the prior literature. For B-domain deleted FVIII constructs, the deletion may also lead to a shift in numeration. For the heavy chain, the numeration correlates with the numeration of the full-length FVIII. From the B-domain deletion on the numeration of the light chain is either kept the same as for the full-length FVIII molecule (e.g. Q763 in front of the deletion is followed by D1582 after the deletion) or can be continued as if no deletion has occurred (e.g., Q763 is followed by D764 despite missing amino acids). The continued numeration complicates the comparison of amino acid sequences, if it is not known how many amino acids were deleted. In the present specification, the numeration of the full-length FVIII molecule is maintained despite (partial) B-domain deletion.

Hemophilia A is a genetic bleeding disorder with deficiency in clotting factor VIII linked to the X-chromosome, occurring in 1 of 5000 newborn males. However, Hemophilia A can also occur spontaneously due to an auto-immune response against FVIII. Patients with Hemophilia A suffer from longer bleeding durations, spontaneous and internal bleedings, affecting their everyday life.

Hemophilia A patients are generally treated by administration of FVIII. Depending on the severity of the disease (mild, moderate or severe) treatment is on demand or prophylactic. Therapeutic FVIII products are either purified from human plasma (pFVIII) or the products are produced recombinantly in cell culture (rFVIII).

During the development of recombinant FVIII molecules for therapy, B-domain deleted FVIII molecules have been designed, because the B-domain is not crucial for the functionality of FVIII in clotting. This predominantly leads to a reduction in size, which facilitates production and storage. The most common B-domain deleted FVIII product is ReFacto® or ReFacto AF® produced by Pfizer. This FVIII variant lacks 894 amino acids of the B domain. However, the furin cleavage recognition site at the end of the B-domain is still present and ReFacto AF® thus is a double chain protein. The review article of Kenneth Lieuw (J Blood Med. 2017; 8: 67-73) highlights some of the differences between Factor VIII products currently available.

Currently, most rFVIII therapeutics are based on the classical double chain FVIII structure comprising the heavy chain (domains A1-a1-A2-a2-[B]) and the light chain (domains a3-A3-C1-C2). During expression, both heavy and light chains are initially translated from one mRNA molecule, but the amino acid sequence comprises a furin cleavage recognition site (R1664-R1667) at the end of the B-domain enabling intracellular proteolytic processing by cleavage after R1667 and separation of both domains. However, heavy and light chains are typically non-covalently connected via a divalent cation.

More recently, recombinant single chain FVIII proteins have been developed. Such B-domain deleted FVIII proteins are designed as a B-domain truncated recombinant FVIII molecule, wherein the light and heavy chains are covalently linked in order to form a stable single chain FVIII molecule (Schmidbauer S. et al., Thromb Res 2015; 136: 388-395). Because according to this approach, the heavy and light chain segments are linked via a strong covalent bond, the segments are less likely to dissociate (Pabinger-Fasching, I., Thromb Res 2016; 141S3: 2-4). The single chain molecules can be isolated as a pure and homogenous compound. It has been observed that the half-life of single chain FVIII molecules was approximately twice that of full-length recombinant FVIII molecules (Zollner S. et al., Thromb Res 2014; 134: 125-131). However, some clinical results with a recombinant, single chain Factor VIII molecule (AFSTYLA®) showed only a marginally enhanced half-life of the single chain molecule compared to a double chain molecule. Nguyen et al. (J Thromb Haemost. 2017 January; 15(1): 110-121) describe novel factor VIII variants with a modified furin cleavage recognition site (furin cleavage recognition site pos. 1664-1667). Variants with mutations at the furin cleavage recognition site are primarily secreted in a single polypeptide chain form and show improved expression compared to double chain B-domain deleted variants.

WO 2017/123961 A1 discloses Factor VIII variants with a B-domain deletion, wherein one or two amino acids at positions 1676 or 1677 are substituted, modified or deleted compared to wild type FVIII. Optionally, the variants may further include a mutated PACE-furin cleavage recognition site (HHQR or RHQR at pos. 1664-1667).

U.S. Pat. No. 6,316,226 B1 discloses a polypeptide having FVIII activity, wherein the polypeptide has an internal deletion of amino acids 760 through 1687 as compared to human FVIII.

WO 2014/008480 A2 discloses single chain FVIII molecules with full or partial deletion of the B domain. A preferred molecule has a deletion of pos. 784-1677 and substitutions R1683A and R1686A (SEQ ID No. 8 in WO 2014/008480 A2).

WO 2013/057219 A1 describes a FVIII molecule wherein a first amino acid selected from the amino acids at positions 760 to 1666 is fused with a second amino acid selected from the amino acids at positions 1668 to 1709. The proteolytic cleavage site between Arg1667 and Glu1668 and, if present, the proteolytic cleavage site between Arg1332 and Ala1333 is inactivated.

WO 2004/067566 A1 discloses FVIII polypeptides comprising an internal deletion of one or more amino acids between 1668 and 1707 fused to any amino acid sequence in B domain from about 760 to 801.

U.S. Pat. No. 6,316,226 B1 discloses a DNA encoding a Factor VIII analog, wherein the analog has an internal deletion of amino acids 760 through 1687 as compared to human Factor VIII.

Further single chain FVIII molecules are described e.g. in WO 88/09813 A1 and WO 2011/041770 A1.

WO 2014/210547 A1, WO 2014/026954 A1, WO 2013/122617 A1, WO 2013/106787 A1, WO 2015/106052 A1 disclose further single chain FVIII variants with modifications or deletions of the furin cleavage recognition site. In summary, single chain variants of FVIII have certain advantages regarding their purification and production, in particular when intended for pharmaceutical use. Purification of wild type FVIII is challenging due to the non-covalent bond between the FVIII heavy and light chains. Furthermore, corresponding non-covalent bonds may also be formed with FVIII fragments. As a result, purification can result in co-purification of fragments and non-stochiometric purification of heavy and light chain, all of which is undesirable for a pharmaceutical product, which ought to be pure, well-defined, and manufactured by a robust process.

However, present single chain FVIII variants suffer from certain disadvantages, such as low expression levels and impaired biological activity. Therefore, there is a need for further and improved FVIII single chain variants with high expression levels and improved biological activity.

In light of the prior art, the inventors addressed the problem of developing improved single chain Factor VIII molecules with an improved and balanced profile of properties. E.g. the molecules allow for easy purification, such as by size exclusion chromatography, and exhibit a high level of expression, while being stable and showing well-retained biological function, including an improved high specific activity.

### SUMMARY OF THE INVENTION

In a first embodiment, the invention provides a recombinant Factor VIII protein comprising, in a single chain, a heavy chain portion comprising an A1 and an A2 domain and a light chain portion comprising an A3, C1 and C2 domain of Factor VIII, wherein,

a) in said recombinant Factor VIII protein, 894 amino acids corresponding to consecutive amino acids between F761 and P1659 of wild type Factor VIII as defined in SEQ ID NO: 1 are deleted, leading to a first deletion;

b) said recombinant Factor VIII protein comprises, spanning the site of the first deletion, a processing sequence comprising SEQ ID NO: 2 or a sequence having at most one amino acid substitution in SEQ ID NO: 2, wherein said processing sequence comprises a first thrombin cleavage site;

c) in said recombinant Factor VIII protein, at least the amino acids corresponding to amino acids R1664 to R1667 of wild type Factor VIII are deleted, leading to a second deletion; and

d) said recombinant Factor VIII protein comprises, C-terminal to the second deletion and N-terminal of the A3 domain, a second thrombin cleavage site.

In a second embodiment, the invention provides a recombinant Factor VIII protein comprising, in a single chain, a heavy chain portion comprising an A1 and an A2 domain and a light chain portion comprising an A3, C1 and C2 domain of Factor VIII, wherein,

a) said recombinant Factor VIII protein comprises a processing sequence comprising SEQ ID NO: 2 or a sequence having at most one amino acid substitution in SEQ ID NO: 2, wherein said processing sequence comprises a first thrombin cleavage site;

b) optionally, directly C-terminal to said processing sequence, said Factor VIII protein comprises a heterologous sequence;

c) directly C-terminal to said processing sequence, or, if present, directly C-terminal to said heterologous sequence, said Factor VIII protein comprises a merging sequence having at least 90% sequence identity to a sequence selected from the group consisting of SEQ ID NO: 9, SEQ ID NO: 10 and SEQ ID NO: 11; and

d) said recombinant Factor VIII protein comprises, C-terminal to SEQ ID NO: 9-11, a second thrombin cleavage site.

In a third embodiment, the Factor VIII protein of the second embodiment (embodiment 2) also is a protein of the first embodiment (embodiment 1).

In a fourth embodiment, the recombinant Factor VIII protein of any of embodiments 1-3, comprises a sequence of SEQ ID NO: 9.

In a fifth embodiment, in the recombinant Factor VIII protein of any of embodiments 1-4, amino acids corresponding to

i) F761 and S1656;

ii) S762 and Q1657;

iii) Q763 and N1658; or

iv) N764 and P1659

of wild type Factor VIII as defined in SEQ ID NO: 1 are adjacent to each other, wherein said amino acids are part of the sequence defined in SEQ ID NO: 2. This is due to the first deletion as defined in the first embodiment.

In a sixth embodiment, in the recombinant Factor VIII protein of any one of embodiments 1-5, the processing sequence is SEQ ID NO: 2 or a sequence having at most one amino acid substitution in said sequence, wherein, optionally, the F, the S C-terminal to the F, the Q or the N are substituted.

In a seventh embodiment, in the recombinant Factor VIII protein of any one of embodiments 1-5, the processing sequence is SEQ ID NO: 3 or a sequence having at most one amino acid substitution in said sequence, wherein, optionally, the F, the S C-terminal to the F, the Q or the N are substituted.

In an eighth embodiment, in the recombinant Factor VIII protein of any one of embodiments 1-5, the processing sequence is SEQ ID NO: 4 or a sequence having at most one amino acid substitution in said sequence, wherein, optionally, the F, the S C-terminal to the F, the Q or the N are substituted.

In a ninth embodiment, in the recombinant Factor VIII protein of any one of embodiments 1-5 or 8, the processing sequence is selected from the group consisting of SEQ ID NO: 4, 5, 6, 7 or 8.

In a tenth embodiment, in the recombinant Factor VIII protein of embodiment 9, the processing sequence is SEQ ID NO: 5. In an eleventh embodiment, in the recombinant Factor VIII protein of embodiment 9, the processing sequence is SEQ ID NO: 6. In a twelfth embodiment, in the recombinant Factor VIII protein of embodiment 9, the processing sequence is SEQ ID NO: 7. In a thirteenth embodiment, in the recombinant Factor VIII protein of embodiment 9, the processing sequence is SEQ ID NO: 8. In a fourteenth embodiment, in the recombinant Factor VIII protein of embodiment 9, the processing sequence is SEQ ID NO: 4.

In a fifteenth embodiment, in the recombinant Factor VIII protein of any one of the preceding embodiments, said processing sequence is directly N-terminal to an amino acid corresponding to Q1675, I1681 or E1690 of wild type Factor VIII.

In a sixteenth embodiment, in the recombinant Factor VIII protein of any one of the preceding embodiments, the amino acids corresponding to amino acids K1663 to L1674 of wild type Factor VIII are deleted, leading to a second deletion.

In a seventeenth embodiment, in the recombinant Factor VIII protein of any one of embodiments 1-16, due to the second deletion, amino acids corresponding to amino acids V1661 and L1674 of wild type Factor VIII as defined in SEQ ID NO: 1 are adjacent to each other, or amino acids corresponding to amino acids L1662 and Q1675 of wild type Factor VIII are adjacent to each other. Both options may result in the same sequence.

In an eighteenth embodiment, in the recombinant Factor VIII protein of any one of embodiments 1-16, due to the second deletion, amino acids corresponding to amino acids V1661 and I1681 of wild type Factor VIII as defined in SEQ ID NO: 1 are adjacent to each other.

In a nineteenth embodiment, in the recombinant Factor VIII protein of any one of embodiments 1-16, due to the second deletion, amino acids corresponding to amino acids P1660 and I1681 of wild type Factor VIII as defined in SEQ ID NO: 1 are adjacent to each other.

In a twentieth embodiment, in the recombinant Factor VIII protein of any one of embodiments 1-16, due to the second deletion, amino acids corresponding to amino acids V1661 and E1690 of wild type Factor VIII as defined in SEQ ID NO: 1 are adjacent to each other.

In a 21^{st} embodiment, the recombinant Factor VIII protein of any one of the preceding embodiments does not comprise a furin cleavage recognition site.

In a 22^{nd} embodiment, the recombinant Factor VIII protein of any one of the preceding embodiments does not comprise a sequence having more than 75%, preferably, more than 50% sequence identity to the furin cleavage recognition site RHQR between the processing sequence and the merging sequence. The sequence corresponding to SEQ ID NO: 15, which comprises said furin cleavage recognition site, may be deleted. Optionally, the recombinant Factor VIII protein of any one of the preceding embodiments does not comprise a sequence having more than 30% sequence identity to SEQ ID NO: 15. Alternatively, it does not comprise a sequence having more than 40% sequence identity to SEQ ID NO: 15.

In a 23^{rd} embodiment, the recombinant Factor VIII protein of any one of the preceding embodiments comprises a processing sequence and, C-terminal to said processing sequence, a merging sequence, wherein the processing sequence is selected from the group comprising SEQ ID NO: 2, 3, 4, 5, 6, 7 or 8, and the merging sequence is selected from the group comprising SEQ ID NO: 12, 13 or 14.

In a 24^{th} embodiment, in the recombinant Factor VIII protein of the 23^{rd} embodiment, the processing sequence is SEQ ID NO: 4 and the merging sequence SEQ ID NO: 12. In a 25^{th} embodiment, in the recombinant Factor VIII protein of the 23^{rd} embodiment, the processing sequence is SEQ ID NO: 5 and the merging sequence SEQ ID NO: 12. In a 26^{th} embodiment, in the recombinant Factor VIII protein of the 23^{rd} embodiment, the processing sequence is SEQ ID NO: 6 and the merging sequence of SEQ ID NO: 12. In a 27^{th} embodiment, in the recombinant Factor VIII protein of the 23^{rd} embodiment, the processing sequence is SEQ ID NO: 7 and the merging sequence of SEQ ID NO: 12. In a 28^{th} embodiment, in the recombinant Factor VIII protein of the 23^{rd} embodiment, the processing sequence is SEQ ID NO: 8 and the merging sequence SEQ ID NO: 12. In a 29^{th} embodiment, in the recombinant Factor VIII protein of the 23^{rd} embodiment, the processing sequence is SEQ ID NO: 3 and the merging sequence of SEQ ID NO: 13. In a 30^{th} embodiment, in the recombinant Factor VIII protein of the 23^{rd} embodiment, the processing sequence is SEQ ID NO: 2 and the merging sequence SEQ ID NO: 13. In a 31^{st} embodiment, in the recombinant Factor VIII protein of the 23^{rd} embodiment, the processing sequence is SEQ ID NO: 3 and the merging sequence SEQ ID NO: 14. In a 32^{nd} embodiment, in the recombinant Factor VIII protein of any one of embodiments 23-31, the merging sequence is directly C-terminal to said processing sequence.

In a 33^{rd} embodiment, the recombinant Factor VIII protein of any one of the preceding embodiments is a fusion protein with at least one heterologous fusion partner selected from the group consisting of an Fc region, an albumin-binding sequence, albumin, PAS polypeptides, HAP polypeptides, the C-terminal peptide of the beta subunit of chorionic gonadotropin, albumin-binding small molecules, polyethylenglycol, hydroxyethyl starch.

In a 34^{th} embodiment, in the recombinant Factor VIII protein of embodiment 33, said heterologous fusion partner is inserted directly C-terminal to said processing sequence. In a 35^{th} embodiment, in the recombinant Factor VIII protein of any of embodiments 33 or 34, said heterologous fusion partner is inserted C-terminal to the C2-domain.

In a 36^{th} embodiment, the recombinant Factor VIII protein of any one of the preceding embodiments further comprises a third thrombin cleavage site between the A1 and A2 domain.

The invention further provides, as a 37^{th} embodiment, a nucleic acid encoding a recombinant Factor VIII protein of any one of the preceding embodiments, wherein said polynucleotide optionally is an expression vector suitable for expression of said recombinant Factor VIII protein in a mammalian cell, e.g., a human cell.

The invention further provides, as a 38^{th} embodiment, a host cell comprising a nucleic acid of embodiment 37, wherein preferably the host cell is a mammalian cell comprising an expression vector suitable for expression of said recombinant Factor VIII protein in said cell. The cell can be a human cell selected from the group comprising a Hek293 cell line or a CAP cell line. Preferably, the cell is a CAP cell line.

The invention further provides, as a 39^{th} embodiment, a method for preparing a Factor VIII protein, comprising culturing the host cell of embodiment 38 under conditions suitable for expression of the Factor VIII protein and isolating the Factor VIII protein, wherein the method optionally comprises formulating the Factor VIII protein as a pharmaceutical composition.

In a 40^{th} embodiment, the invention provides a composition comprising recombinant Factor VIII protein of any one of embodiments 1-36, wherein the content of single chain protein Factor VIII protein of all Factor VIII protein is at least 90%.

The invention further provides, as a 41^{st} embodiment, a pharmaceutical composition comprising the recombinant Factor VIII protein of any of embodiments 1-36 or 40, the nucleic acid of embodiment 37, or the host cell of embodiment 38. The pharmaceutical composition may comprise a pharmaceutically acceptable solvent, e.g., water or a buffer, and/or pharmaceutically acceptable excipients. In a 42^{nd} embodiment, the pharmaceutical composition of embodiment 41, or a kit comprising the pharmaceutical composition of embodiment 41, further comprises an immunosuppressive agent, e.g., immunosuppressive agent selected from the group comprising methylprednisolone, prednisolone, dexamethason, cyclophosphamide, rituximab, and/or cyclosporin.

The invention further provides, as a 43^{rd} embodiment, a pharmaceutical composition of any of embodiments 41 or 42 for use in treatment of hemophilia A, wherein, optionally, the treatment is immune tolerance induction (ITI). In a 44^{th} embodiment, the pharmaceutical composition of any of embodiments 41-43 is for use in treating a patient with Hemophilia A selected from the group comprising a patient not previously treated with any Factor VIII protein, a patient previously treated with a Factor VIII protein, a patient who has an antibody response including an inhibitory antibody response to a Factor VIII protein, and a patient who has had an antibody response including an inhibitory antibody response to a Factor VIII protein who has been treated by ITI, or who has not been treated by ITI.

In a 45^{th} embodiment, the invention provides a vial, e.g., a prefilled or ready-to use syringe, comprising the pharmaceutical composition of any of embodiments 41-44.

In a 46^{th} embodiment, the invention provides a method of treatment, comprising administering an effective amount of the pharmaceutical composition of any of embodiments 41-44 to a patient in need thereof, e.g., a patient with hemophilia A, which may be selected from the patient groups defined herein.

### FIGURE LEGENDS

FIG. 1 shows comparative single chain FVIII protein constructs in which the furin cleavage recognition site was deleted. Variants AC_SC-V1 (V1) and -V3 (V3) have a natural thrombin cleavage site (PR/SV or SC/SV, respectively), and there is only a single deletion of aa (amino acid/s) 760-1687 (V1) and aa 731-1687 (V3). Variants AC_SC-V2 (V2) and -V4 (V4), compared to V1 and V2, comprise a different thrombin cleavage site (PR/VA or IR/SV, respectively), wherein V2, based on V1, comprises a VA sequence which can be considered as being derived from the B-domain. V4, based on V3, comprises an insertion DPR-IRSV-VAQ at the site of the deletion.

FIG. 2 shows wt FVIII (A) and single chain FVIII protein constructs AC_SC-V0 (B), -V5 (C), -V6 (D), and -V7 (E) (or, short, V0, V5, V6, V7), which are based on the ReFacto AF® amino acid sequence AC-6rs-Ref SC, by introduction of two deletions. Arrows show thrombin cleavage sites. Sig is the signal peptide (19 aa). Bold and italic letters indicate the amino acids of the thrombin cleavage recognition site with cleavage after aa 759 (i.e., in the processing sequence in the constructs of the invention). The furin cleavage recognition site is underlined in the wildtype protein. The italic numbers in parenthesis relate to the amino acid numbers in the wild type sequence.

FIG. 3 Comparison of unpurified single chain FVIII variants for their in vitro functionality. Cell culture supernatants of CAP-T cells expressing the double chain FVIII molecule AC-6rs-REF, and the single chain FVIII variants AC_SC-V0, -V1, -V2, -V5, -V6, -V7 were analysed for chromogenic FVIII activity (A), FVIII clotting activity induced by Actin FSL (B), FVIII antigen levels indicating total FVIII protein amount (C). Specific chromogenic activity was calculated as chromogenic FVIII activity to FVIII antigen ratio displayed in % (D). Specific clotting activity was calculated as clotting FVIII activity to FVIII antigen ratio displayed in % (E). n=2.

FIG. 4 shows a comparison of stability (as determined by chromogenic activity) of V0 and ReFacto AF® in vitro over 24 h (A) and over 14 days (B). In A, stability is analysed in buffer and ReFacto AF® is shown with diamonds and V0 (AC-SC) with squares. In B, ReFacto AF® in FVIII formulation buffer is shown, with diamonds, and in FVIII-depleted plasma (FVIII-dp), with squares, and V0 in buffer, with triangles, and in FVIII-dp, with crosses.

FIG. 5 shows the regression curve of normalized activity (as determined by chromogenic activity) of V0 (continuous line) and ReFacto AF® (dashed line) as a result of a non-compartment analysis from an in vivo pharmacokinetic study in Hemophila A mice.

### SEQUENCES

SEQ ID NO: 1 wild type Factor VIII

SEQ ID NO: 2 PRSFSQNPP minimal processing sequence

SEQ ID NO: 3 PRSFSQNPPV processing sequence

SEQ ID NO: 4 PRSFSQNPPVL processing sequence

SEQ ID NO: 5 PRSXSQNPPVL processing sequence

SEQ ID NO: 6 PRSFXQNPPVL processing sequence

SEQ ID NO: 7 PRSFSXNPPVL processing sequence

SEQ ID NO: 8 PRSFSQXPPVL processing sequence

SEQ ID NO: 9 QSDQEEIDYD, merging sequence, e.g., in V0

SEQ ID NO: 10 IDYDDTI merging sequence, e.g., in V5+V6

SEQ ID NO: 11 EMKKEDFD merging sequence, e.g., in V7

SEQ ID NO: 12 Q1675 to R1708 von V0, merging sequence, e.g., in V0

SEQ ID NO: 13 I1681 to R1708 von V6, merging sequence, e.g., in V6

SEQ ID NO: 14 E1690 to R1708 von V7, merging sequence, e.g., in V7

SEQ ID NO: 15 KRHQREITRTT sequence comprising furin cleavage recognition site deleted in proteins of the invention

SEQ ID NO: 16 V0 aa sequence

SEQ ID NO: 17 V1 aa sequence

SEQ ID NO: 18 V2 aa sequence

SEQ ID NO: 19 V3 aa sequence

SEQ ID NO: 20 V4 aa sequence

SEQ ID NO: 21 V5 aa sequence

SEQ ID NO: 22 V6 aa sequence

SEQ ID NO: 23 V7 aa sequence

SEQ ID NO: 24 V0 na sequence

SEQ ID NO: 25 V5 na sequence

SEQ ID NO: 26 V6 na sequence

SEQ ID NO: 27 V7 na sequence

SEQ ID NO: 28 AC-6rs-REF aa sequence

SEQ ID NO: 29 AC-6rs aa sequence

SEQ ID NO: 30 AC-6rs-REF na sequence

SEQ ID NO: 31 SVEMKKEDF merging sequence in V1

SEQ ID NO: 32 DSYEDISAYLLSKNNAIEPR sequence N-terminal to processing sequence and including 2 aa of processing sequence, i.e., sequence N-terminal of thrombin cleavage site

SEQ ID NO: 33-38 partial FVIII sequences

### DETAILED DESCRIPTION OF THE INVENTION

In a first embodiment, the invention provides a recombinant Factor VIII protein comprising, in a single chain, a heavy chain portion comprising an A1 and an A2 domain and a light chain portion comprising an A3, C1 and C2 domain of Factor VIII, wherein,

a) in said recombinant Factor VIII protein, 894 amino acids corresponding to consecutive amino acids between F761 and P1659 of wild type Factor VIII as defined in SEQ ID NO: 1 are deleted, leading to a first deletion;

b) said recombinant Factor VIII protein comprises, spanning the site of the first deletion, a processing sequence comprising SEQ ID NO: 2 or a sequence having at most one amino acid substitution in SEQ ID NO: 2, wherein said processing sequence comprises a first thrombin cleavage site;

c) in said recombinant Factor VIII protein, at least the amino acids corresponding to amino acids R1664 to R1667 of wild type Factor VIII are deleted, leading to a second deletion; and d) said recombinant Factor VIII protein comprises, C-terminal to the second deletion and N-terminal of the A3 domain, a second thrombin cleavage site.

The inventors have construed new FVIII single chain variants in which the region comprising a deletion of the B-domain is shorter than in other single-chain FVIII variants. In particular, by retaining an internal fragment of the B-domain, the inventors have managed to form a processing sequence (see SEQ ID NO. 2) which is closer to the wild type processing sequence. Preferably, part of the processing sequence corresponds to a truncated B-domain, thus the processing sequence is embedded into a sequence derived from wild type FVIII. As a consequence of these findings, the resulting FVIII proteins show a high level of expression, a low profile of fragments and side products, and in particular a high specific activity as evidenced by different biological activity assays. Furthermore, the inventors have found certain amniocyte cell lines to be particularly well suited for high-level expression of functional molecules.

Further advantages and preferred embodiments are explained elsewhere in this description.

Side-by-side comparisons with either double-chain FVIII (using the Moroctocog Alfa sequence, see Examples) or single-chain variants of the state of the art (variants V1 and V2, see Examples), show high expression levels combined with improved specific activity. The FVIII proteins according to the invention produce also much better results than other single chain variants designed and tested (V3, V4).

The skilled person understands the term FVIII (or Factor VIII) and is aware of the structure and biological functions of wild type FVIII and typical variants thereof. Apart from the sequences specified above, the FVIII protein of the invention may be designed as deemed appropriate and advantageous by the skilled person. In particular, the Factor VIII protein of the invention should typically comprise all necessary portions and domains known to be important for biological function. For example, preferably, the FVIII protein further comprises domains corresponding to, substantially corresponding to, and/or functionally corresponding to the A and C domains of wild type FVIII. It may further comprise additional portions and domains. For example, preferably, the FVIII protein further comprises an a1 domain between the A1 and the A2 domains and an a2 domain C-terminal to the A2 domain, wherein the processing sequence is located at the C-terminal end of the a2 domain. Part of the processing sequence corresponds to a truncated B-domain. C-terminal to said processing sequence, the FVIII protein comprises at least a truncated a3 domain, which may comprise a merging sequence as defined herein. Before processing, the Factor VIII protein of the invention may also comprise a signal sequence. Any or all of said domains may be wildtype (wt) FVIII domains, or they may differ from the wildtype domains, e.g., as known in the state of the art or deemed appropriate by the skilled person. The domains are preferably contained in the protein in that order, i.e., from N-terminus to C-terminus of the protein.

A FVIII protein according to the present invention shall have at least one biological activity or function of a wt FVIII protein, in particular with regard to the function in coagulation. The FVIII protein should be cleavable by thrombin, leading to activation. Preferably, said thrombin recognition and/or thrombin cleavage sites correspond to or substantially correspond to those of wild type FVIII. It is then capable of forming a complex with the activated coagulation Factor IXa and coagulation Factor X, and the light chain is capable of binding to a phospholipid bilayer, e.g., the cell membrane of (activated) platelets.

The biological activity of FVIII can be determined by analyzing the chromogenic or the coagulant activity of the protein, as described herein. Typically, the chromogenic activity is taken as a measure of biological activity.

The other parts of the FVIII protein of the invention can be designed as desired by the skilled person, but preferably maintaining a high FVIII biological activity. As shown in the Examples, the invention allows to generate a FVIII protein with a high biological activity, as measured e.g. by the chromogenic activity. Therefore, preferably the FVIII protein according to the invention has a chromogenic activity which is at least comparable to the activity of the wt protein, i.e., it has at least 50% of the chromogenic activity of the wt protein (SEQ ID NO: 1). Preferably, the FVIII protein according to the invention has at least 80%, at least 100% or more than 100% of the chromogenic activity of the wt protein. Preferably, the chromogenic activity also is at least 80%, at least 90%, at least 100% or more than 100% of the chromogenic activity of ReFacto AF® (international non-proprietary name: Moroctocog Alfa), a commercially available B-domain deleted FVIII (Pfizer).

As defined in a), in the FVIII of the invention, 894 amino acids corresponding to consecutive amino acids between F761 and P1659 of wild type Factor VIII as defined in SEQ ID NO: 1 are deleted in the Factor VIII protein of the invention, leading to a first deletion. In certain embodiments, in particular, starting from a numbering of amino acids in FVIII without deletions or insertions, the term “corresponding to” should be understood to mean “identical to”.

For specific amino acids which may be mutated compared to the wt, an amino acid corresponding to the wild type aa is determined by an alignment e.g. using EMBOSS Needle (based on the Needleman-Wunsch algorithm; settings: MATRIX: “BLOSUM62”, GAP OPEN: “20”, GAP EXTEND:“0.5”, END GAP PENALTY: “false”, END GAP OPEN: “10”, END GAP EXTEND: “0.5”).

In order to assess sequence identities of two polypeptides, such an alignment may be performed in a two-step approach: I. A global protein alignment is performed using EMBOSS Needle (settings: MATRIX: “BLOSUM62”, GAP OPEN: “20”, GAP EXTEND:“0.5”, END GAP PENALTY: “false”, END GAP OPEN: “10”, END GAP EXTEND: “0.5”) to identify a particular region having the highest similarity. II. The exact sequence identity is defined by a second alignment using EMBOSS Needle (settings: MATRIX: “BLOSUM62”, GAP OPEN: “20”, GAP EXTEND:“0.5”, END GAP PENALTY: “false”, END GAP OPEN: “10”, END GAP EXTEND: “0.5”) comparing the fully overlapping polypeptide sequences identified in (I) while excluding non-paired amino acids.

“between” excludes the recited amino acids, e.g., it means that the recited amino acids are maintained. “deletion” or “deleted” does not necessitate that the protein was actually prepared by deleting amino acids previously present in a predecessor molecule, but it merely defines that the amino acids are absent, independent from the preparation of the molecule. For example, the protein can be produced based on nucleic acids prepared by de novo synthesis or by genetic engineering techniques.

As defined in b), the recombinant Factor VIII protein comprises, spanning the site of the first deletion, a processing sequence comprising SEQ ID NO: 2 (PRSFSQNPP) or a sequence having at most one amino acid substitution in SEQ ID NO: 2, wherein said processing sequence comprises a first thrombin cleavage site. Accordingly, at least one amino acid of the processing sequence corresponds to an amino acid C-terminal to the deletion and at least one amino acid of the processing sequence corresponds to an amino acid N-terminal to the deletion. The processing sequence comprises SEQ ID NO: 2 or a sequence having at most one amino acid substitution in SEQ ID NO: 2, i.e., the processing sequence can be longer. In particular, the processing sequence is selected from the group comprising SEQ ID NO: 2, 3, 4, 5, 6, 7 or 8. The inventors have found that a processing sequence of the invention enables a particularly good cleavage by thrombin.

Typically, the processing sequence is not longer than SEQ ID NO: 4. The processing sequence may be directly C-terminal to sequences from the a2 domain, e.g., wt a2 domain sequences. The first N-terminal two amino acids of the processing sequence may already belong to the a2 domain. Preferably, the amino acid directly N-terminal to the processing sequence is E.

One amino acid in SEQ ID NO: 2 can be substituted, e.g., to reduce immunogenicity. Optionally, the F, the S C-terminal to the F, the Q or the N are substituted.

The processing sequence may be SEQ ID NO: 2 or a sequence having at most one amino acid substitution in said sequence, wherein, optionally, the F, the S C-terminal to the F, the Q or the N are substituted. For example, the FVIII proteins of the invention V0, V5, V6 and V7 comprise SEQ ID NO: 2. The processing sequence of V6 consists of SEQ ID NO: 2.

The processing sequence may alternatively be SEQ ID NO: 3 (PRSFSQNPPV) or a sequence having at most one amino acid substitution in said sequence, wherein, optionally, the F, the S C-terminal to the F, the Q or the N are substituted. For example, the FVIII proteins of the invention V0, V5, and V7 comprise SEQ ID NO: 3. The processing sequence of V5 and V7 consists of SEQ ID NO: 3.

The processing sequence may alternatively be SEQ ID NO: 4 (PRSFSQNPPVL) or a sequence having at most one amino acid substitution in said sequence, wherein, optionally, the F, the S C-terminal to the F, the Q or the N are substituted. For example, the present inventors have shown that an L at the C-terminus of the processing sequence, as in SEQ ID NO: 4, 5, 6, 7 or 8, endows the FVIII with particularly good activity. The processing sequence of the FVIII protein of the invention V0, which has been found to be particularly advantageous, consists of SEQ ID NO: 4, which is a specific embodiment of SEQ ID NO: 5-8.

The alternative processing sequences SEQ ID NO: 5 (PRSXSQNPPVL), SEQ ID NO: 6 (PRSFXQNPPVL), SEQ ID NO: 7 (PRSFSXNPPVL) and SEQ ID NO: 8 (PRSFSQXPPVL) are variants in which X can be any naturally occurring amino acid. Optionally, X is a conservative substitution compared to the corresponding amino acid in SEQ ID NO: 4, i.e. a hydrophobic amino acid is substituted by a hydrophobic amino acid, a hydrophilic amino acid is substituted by a hydrophilic amino acid, an aromatic amino acid by an aromatic amino acid, an acid amino acid by an acid amino acid and a basic amino acid by a basic amino acid.

As defined in c), in the FVIII protein of the invention, the amino acids corresponding to amino acids R1664 to R1667 of wild type Factor VIII are deleted, leading to a second deletion. These amino acid correspond to the furin cleavage recognition site of wt FVIII. Accordingly, the protein is essentially not cleaved by furin. In a composition, at least 80%, optionally, at least 90% or at least 95% of the FVIII protein of the invention are present in a single chain form.

As defined in d), the recombinant Factor VIII protein of the invention comprises, C-terminal to the second deletion and N-terminal of the A3 domain, a second thrombin cleavage site. Accordingly, upon activation, the part of the FVIII protein between the thrombin cleavage site in the processing sequence and the second thrombin cleavage site are excised from the activated FVIII protein.

As a second embodiment, the invention also provides a recombinant Factor VIII protein comprising, in a single chain, a heavy chain portion comprising an A1 and an A2 domain and a light chain portion comprising an A3, C1 and C2 domain of Factor VIII, wherein,

a) said recombinant Factor VIII protein comprises a processing sequence comprising SEQ ID NO: 2 or a sequence having at most one amino acid substitution in SEQ ID NO: 2, wherein said processing sequence comprises a first thrombin cleavage site;

b) optionally, directly C-terminal to said processing sequence, said Factor VIII protein comprises a heterologous sequence;

c) directly C-terminal to said processing sequence, or, if present, directly C-terminal to said heterologous sequence, said Factor VIII protein comprises a merging sequence having at least 90% sequence identity to a sequence selected from the group consisting of SEQ ID NO: 9 (QSDQEEIDYD), SEQ ID NO: 10 (IDYDDTI) and SEQ ID NO: 11 (EMKKEDFD); and

d) said recombinant Factor VIII protein comprises, C-terminal to SEQ ID NO: 9-11, a second thrombin cleavage site.

Said Factor VIII protein optionally is a Factor VIII protein as described above as the first embodiment. In any case, the definitions provided herein apply to both embodiments.

As described in b), optionally, directly C-terminal to the processing sequence, said Factor VIII protein comprises a heterologous sequence. A heterologous sequence does not occur in the wild type protein at the same relative location, and it optionally does not consist of the same number of amino acids as a sequence deleted from the protein of the invention. Preferably, a heterologous sequence comprises a non-FVIII sequence of at least 10, optionally, at least 20, at least 30 or at least 40 amino acids that do not occur in wild type FVIII, and, preferably, has less than 30% sequence identity to any wildtype FVIII fragment, optionally, less than 25% sequence identity to any wildtype FVIII fragment. In particular, it may have less than 20% sequence identity to any wt FVIII sequence from the B-domain or the a3 domain. A heterologous sequence may however comprise subsequences which are found in FVIII. Optionally, at least 60% of the heterologous sequence are non-FVIII sequences.

As described in c), directly C-terminal to the processing sequence, or, if present, directly C-terminal to said heterologous sequence, said Factor VIII protein comprises a merging sequence having at least 90% sequence identity to a sequence selected from the group consisting of SEQ ID NO: 9 (QSDQEEIDYD), SEQ ID NO: 10 (IDYDDTI) and SEQ ID NO: 11 (EMKKEDFD). The term “merging sequence” illustrates that the sequence is merging into the final sequence together with the processing sequence, and, optionally, directly C-terminal to said sequence, which can be effected through a deletion in wt FVIII. The merging sequence may also be designated “a3 derived” sequence, which illustrates the origin of the sequence. It does not necessarily mean that the sequence corresponds to all a3 sequences comprised in the recombinant Factor VIII protein of the invention. Preferably, there are no a3-derived amino acids N-terminal to the defined merging sequence, but, in particular for SEQ ID NO: 9-11, there may be a3-derived amino acids C-terminal to said defined sequence, e.g., as defined in SEQ ID NO: 12, 13 or 14.

Preferably, the Factor VIII protein may comprise a merging sequence selected from the group consisting of SEQ ID NO: 9, 10 or 11. The inventors could show that it is advantageous if the FVIII protein comprises a longer sequence corresponding to parts of the a3 domain which are C-terminal of SEQ ID NO: 9, preferably resulting in the sequence of SEQ ID NO: 12. The same applies to SEQ ID NO: 10, preferably resulting in the sequence of SEQ ID NO: 13 and to SEQ ID NO: 11, preferably resulting in the sequence of SEQ ID NO: 14. Accordingly, preferably, the recombinant Factor VIII protein comprises SEQ ID NO: 9. For example the V0 protein comprises SEQ ID NO: 9. It also comprises SEQ ID NO: 10 and SEQ ID NO: 11, which are derived from the a3-region (at least partly) C-terminal to SEQ ID NO: 9. V5 and V6 comprise SEQ ID NO: 10 and V7 comprises SEQ ID NO: 11.

The Factor VIII proteins of the invention can also be defined insofar as amino acids corresponding to

i) F761 and S1656;

ii) S762 and Q1657;

iii) Q763 and N1658; or

iv) N764 and P1659

of wild type Factor VIII as defined in SEQ ID NO: 1 are adjacent to each other, wherein said amino acids are part of the sequence defined in SEQ ID NO: 2. This is due to the first deletion as defined in the first embodiment. The skilled person will note that this particular construction leads to maintenance of amino acids identical without the deletion and with the deletion. Thus, the structure of the protein may be less affected by the deletion than with other deletions of the B-domain known in the state of the art. “adjacent” in the context of the invention means the same as “immediately adjacent”, and relates to the secondary structure of proteins.

In one embodiment of the invention, wherein no heterologous sequence is inserted directly C-terminal to the processing sequence, it is further preferred that the processing sequence is directly N-terminal to an amino acid corresponding to Q1675, I1681 or E1690 of wild type Factor VIII. For example, this occurs for Q1675 in V0, for I1681 in V5 and V6 and for E1690 in V7.

In a Factor VIII protein of the invention, the 7 N-terminal amino acids of the a3-domain are preferably absent (i.e., deleted), i.e., the a3-domain is partially deleted or truncated.

In a Factor VIII protein of the invention, the amino acids corresponding to amino acids K1663 to L1674 of wild type Factor VIII may be deleted, leading to a second deletion. This is the case, e.g., in V0 and V5-V7.

In FVIII proteins of the invention, due to the second deletion, amino acids corresponding to amino acids V1661 and L1674 of wild type Factor VIII as defined in SEQ ID NO: 1 may be adjacent to each other, or amino acids corresponding to amino acids L1662 and Q1675 of wild type Factor VIII may be adjacent to each other. V0 is an example of a protein for which this applies. In case a heterologous sequence is inserted directly C-terminal to the processing sequence, the amino acids corresponding to amino acids L1662 and Q1675 of wild type Factor VIII are not adjacent to each other.

In other FVIII proteins of the invention, e.g., V5, due to the second deletion, amino acids corresponding to amino acids V1661 and I1681 of wild type Factor VIII as defined in SEQ ID NO: 1 are adjacent to each other.

In other FVIII proteins of the invention, e.g., V6, due to the second deletion, amino acids corresponding to amino acids P1660 and I1681 of wild type Factor VIII as defined in SEQ ID NO: 1 are adjacent to each other.

In other FVIII proteins of the invention, e.g., V7, due to the second deletion, amino acids corresponding to amino acids V1661 and E1690 of wild type Factor VIII as defined in SEQ ID NO: 1 are adjacent to each other.

The recombinant Factor VIII protein of the invention does not comprise a furin cleavage recognition site. Preferably, it also does not comprise a sequence having more than 75% sequence identity to the furin cleavage recognition site RHQR between the processing sequence and the merging sequence. A deletion of the furin cleavage recognition site has been found to be superior to other mutations, e.g., substitutions. Optionally, the recombinant Factor VIII protein of the invention does not comprise a sequence having more than 50% sequence identity to the furin cleavage recognition site RHQR between the processing sequence and the merging sequence.

Preferably, other amino acids in the vicinity of the furin cleavage recognition site are also deleted. Accordingly, preferably, the FVIII proteins of the invention do not comprise a sequence having more than 30% sequence identity to SEQ ID NO: 15 (KRHQREITRTT, i.e. aa K1663-T1673 of SEQ ID NO: 1), which comprises the furin cleavage recognition site. Optionally, they do not comprise a sequence having more than 40% sequence identity to SEQ ID NO: 15.

As the furin cleavage recognition site is not present, the protein is essentially not cleaved by furin, and the content of single chain protein Factor VIII protein of all Factor VIII protein is at least 90%.

Particularly good results regarding expression and activity have been found for recombinant Factor VIII proteins of the invention, comprising a processing sequence and, C-terminal to said processing sequence, a merging sequence, wherein the processing sequence is selected from the group comprising SEQ ID NO: 2, 3, 4, 5, 6, 7 or 8, and the merging sequence is selected from the group comprising SEQ ID NO: 12, 13 or 14.

Therefore, the invention also provides recombinant Factor VIII proteins comprising a processing sequence and, C-terminal to said processing sequence, a merging sequence, wherein the processing sequence is selected from the group comprising SEQ ID NO: 2, 3, 4, 5, 6, 7 or 8, and the merging sequence is selected from the group comprising SEQ ID NO: 12, 13 or 14.

For example, the processing sequence may be SEQ ID NO: 4 and the merging sequence SEQ ID NO: 12, as, e.g., in construct V0. The processing sequence may also be SEQ ID NO: 5 and the merging sequence SEQ ID NO: 12. The processing sequence may be SEQ ID NO: 6 and the merging sequence SEQ ID NO: 12. The processing sequence may be SEQ ID NO: 7 and the merging sequence SEQ ID NO: 12. The processing sequence may be SEQ ID NO: 8 and the merging sequence SEQ ID NO: 12. The processing sequence may be SEQ ID NO: 3 and the merging sequence SEQ ID NO: 13, as, e.g., in V5. The processing sequence may be SEQ ID NO: 2 and the merging sequence SEQ ID NO: 13, as, e.g., in V6. The processing sequence may be SEQ ID NO: 3 and the merging sequence SEQ ID NO: 14, as, e.g., in V7. Optionally, said merging sequence is directly C-terminal to said processing sequence. Alternatively, a heterologous sequence may be inserted between the processing sequence and the merging sequence, e.g., as defined for the fusion partners below.

The recombinant Factor VIII protein of the invention typically further comprises a third thrombin cleavage site between the A1 and A2 domain. It may also comprise further thrombin cleavage sites, as long as the biological function is maintained, but this is not required.

Preferred FVIII proteins of the invention comprise the amino acid sequence of the mature protein (i.e., without signal sequence) of any of SEQ ID NO: 16 (V0), 21 (V5), 22 (V6) or 23 (V7), preferably, SEQ ID NO: 16, or a fusion protein of any of these proteins.

Thus, the invention provides recombinant Factor VIII molecules, comprising an amino acid sequence according to SEQ ID NO: 16, SEQ ID NO: 21, SEQ ID NO: 22, or SEQ ID NO: 23 (each without the signal sequence), or which are fusion proteins comprising at least one of these sequences. A protein comprising the amino acid sequence according to SEQ ID NO: 16 (without the signal sequence) has been shown to have particularly good characteristics. The signal sequence corresponds to amino acids 1-19 of the respective proteins. In the mature protein, which is typically used, in particular, for pharmaceutical purposes, the signal sequence is normally missing. However, it is also possible that the signal sequence is included in the protein of the invention.

A fusion partner may be employed to extend the in vivo plasma half-life of the FVIII protein of the invention. In one embodiment, the recombinant Factor VIII protein of the invention is a fusion protein with at least one heterologous fusion partner, preferably with a fusion partner extending the in vivo plasma half-life of the FVIII protein. The fusion partner may e.g. be selected from the group comprising an Fc region, albumin, an albumin binding sequence, PAS polypeptides, HAP polypeptides, the C-terminal peptide of the beta subunit of chorionic gonadotropin, albumin-binding small molecules, and combinations thereof. The FVIII protein may alternatively or additionally be covalently linked to non-protein fusion partners such as PEG (polyethylenglycol) and/or HES (hydroxyethyl starch). PAS polypeptides or PAS sequences are polypeptides comprising an amino acid sequence comprising mainly alanine and serine residues or comprising mainly alanine, proline and serine residues, the PAS sequences forming a random coil conformation under physiological conditions, as defined in WO 2015/023894. HAP polypeptides or sequences are homo-amino acid polymer (HAP), comprising e.g., repetitive sequences of Glycine or Glycine and Serine, as defined in WO 2015/023894. Potential fusions, fusion partners and combinations thereof are described in more detail e.g., in WO 2015/023894.

Optionally, for certain therapeutic applications, the recombinant FVIII protein may be fused to an Fc region. A fusion to an Fc region may be used to extend the half-life and reduce immunogenicity.

The inventors have found that said heterologous fusion partner may advantageously be inserted directly C-terminal to said processing sequence and/or C-terminal to the C2-domain. These locations have been found by the inventors to be advantageous for fusion, while maintaining good biological activity of the FVIII protein. Optionally, the fusion protein further comprises at least one linker.

The protein may further be glycosylated and/or sulfated. Preferably, post-translational modifications such as glycosylation and/or sulfation of the protein occur in a human cell. A particularly suitable profile of post-translational modifications can be achieved using CAP cells, in particular CAP-T cells or CAP-Go cells (WO 2001/36615; WO 2007/056994; WO 2010/094280; WO 2016/110302). CAP cells, available from Cevec Pharmaceuticals GmbH (Cologne, Germany), originate from human amniocytes as they were isolated trans-abdominally during routine amniocentesis. Obtained amniocytes were transformed with adenoviral functions (E1A, E1B, and pIX functions) and subsequently adapted to growth in suspension in serum-free medium.

wt FVIII typically is bound by vWF. vWF can shield against degradation and may have a positive impact on immune tolerance. Therefore, in certain embodiments of the invention, the protein should be capable of association with vWF. For example, the binding potency of the FVIII protein of the invention to vWF is 10%-100%, 10%-90%, 20-80%, 30-70%, 40-60% or 50-60% of the binding potency of ReFacto AF® to vWF, which can be determined by an ELISA-based method. vWF binding is mediated in particular by amino acid positions Y1683 and Y1699. These should not be mutated if vWF binding is desired.

In certain cases, it may be the case or even desirable that the FVIII protein is not capable of association with vWF. This may, for example, be the case if stabilization is mediated by different means than vWF binding. To avoid vWF binding, e.g. amino acids Y1683 and/or Y1699 may be mutated, or vWF binding may be sterically hindered by a different binding partner.

Advantageously, the FVIII protein according to the invention is capable of being sufficiently stable for pharmaceutical use. The inventors could show that stability of FVIII proteins of the invention in vitro and in vivo is comparable to stability of ReFacto AF®, see Examples. Therefore, the protein of the invention is preferably sufficiently stable in human plasma in vitro and/or in vivo, particularly in vivo. Preferably, in vivo, the half-life of the FVIII protein of the invention in human plasma (in a patient without inhibitors) is about at least 6 hours, preferably, at least 12 hours, at least 18 hours, at least 24 hours, or at least 30 hours. As defined herein, the FVIII protein may be a FVIII protein without fusion partner, or it may be a fusion protein as defined herein. However, as shown in the Examples, the specified half-life can already be obtained without fusion partners. In case of the presence of one or more fusion partners, the half-life of the FVIII protein may be the same, or even longer.

The invention also provides a nucleic acid encoding a recombinant Factor VIII protein of the invention. Said polynucleotide may be an expression vector, e.g., suitable for expression of said recombinant Factor VIII protein in a mammalian cell, such as a human cell.

The nucleic acid preferably encodes the FVIII with an N-terminal signal sequence, e.g., the 19 aa signal sequence of SEQ ID NO: 1. Preferred nucleic acids of the invention encode SEQ ID NO: 16 and 21-23 (V0, V5, V6, V7), or, optionally, a fusion protein thereof. They may be SEQ ID NO: 24-27. The nucleic acids of the invention may be DNA molecules or RNA molecules. The nucleic acids may be optimized for expression in the host cell, e.g., in a human cell.

The expression vector comprises the sequence encoding the FVIII protein, preferably, in codon-optimized form, under the functional control of a suitable promoter, which may be a constitutive or an inducible promoter. The promoter may be a promoter not associated with expression of FVIII in nature, e.g., EF-1alpha or a heterologous promoter, e.g., CMV or SV40. It may further comprise pro- and/or eukaryotic selection markers, such as ampicillin resistance and dihydrofolate reductase (dhfr), and origins of replication, e.g., an SV40 origin and/or a pBR322 origin. “codon-optimized” means optimized for expression in the host cell, preferably, for expression in a human host cell.

Alternatively, the nucleic acid may be a vector suitable for gene therapy, e.g., for gene therapy of a human patient. Vectors suitable for gene therapy are known in the art, e.g., virus-based vectors e.g., based on adenovirus or adeno-associated virus (AAV) or based on retrovirus, such as lentiviral vectors etc. or non virus-based vectors such as but not limited to small plasmids and minicircles or transposon-based vectors. An AAV-based vector of the invention may e.g., be packaged in AAV particles for gene therapy of Hemophilia A patients.

The invention also provides a host cell comprising a nucleic acid of the invention. The host cell may be a bacterial cell, a plant cell, a fungal cell, a yeast cell or an animal cell. Preferably, the host cell is an animal cell, in particular, a mammalian cell comprising an expression vector suitable for expression of said recombinant Factor VIII protein in said cell. The host cell preferably is a human cell comprising an expression vector suitable for expression of said recombinant Factor VIII protein in said human cell. The cell may be transiently or stably transfected with the nucleic acid of the invention. The cell may be a cell line, a primary cell or a stem cell. For production of the protein, the cell typically is a cell line such as a HEK cell, such as a HEK-293 cell, a CHO cell, a BHK cell, a human embryonic retinal cell such as Crucell's Per.C6 or a human amniocyte cell such as CAP. For treatment of human patients with the protein, the host cell preferably is a human cell, e.g., a HEK293 cell line or a CAP cell line (e.g. a CAP-T cell or a CAP-Go cell). The inventors have found that in a CAP cell line, a particularly high single chain content of FVIII protein of the invention is produced. Among the CAP cells, CAP-T cells are preferred for transient expression, while CAP-Go cells may be used for creation of stable cell lines conveying an advantageous glycosylation profile to the FVIII molecule.

The cell may be an autologous cell of a Hemophilia A patient suitable for producing FVIII in the patient after transfection and reintroduction into the patient's body. The cell may be a stem cell, e.g., a hematopoietic stem cell, but preferably it is not an embryonic stem cell, in particular when the patient is a human. The cell may also be hepatocyte, a liver sinusoidal endothelial cell or a thrombocyte.

Cell lines expressing the protein of the invention may also be used in a method of preparing the protein of the invention, comprising cultivating said cells under conditions suitable for expression of the FVIII protein and purifying said protein, e.g., using a plurality of methods known to the skilled person, e.g., as described herein. Such purification methods may comprise standard harvesting procedures for cell removal, e.g. centrifugation, followed by chromatography steps, e.g. affinity chromatography, and methods for exchanging the FVIII proteins into a suitable buffer. The invention thus also provides a method for preparing a Factor VIII protein, comprising culturing the host cell of the invention under conditions suitable for expression of the Factor VIII protein and isolating the Factor VIII protein, wherein the method optionally comprises formulating the Factor VIII protein as a pharmaceutical composition.

The invention thus provides a pharmaceutical composition comprising the recombinant Factor VIII protein of the invention, the nucleic acid of the invention or the host cell of the invention. Such pharmaceutical compositions may comprise suitable excipients, e.g., a buffer, a stabilizing agent, a bulking agent, a preservative, another (e.g., recombinant) protein or combinations thereof. In the context of the invention, if not explicitly stated otherwise, “a” is understood to mean one or more. A suitable buffer for formulation may e.g. contain 205 mM NaCl, 5.3 mM CaCl₂, 6.7 mM L-Histidine, 1.3% Sucrose and 0.013% Tween 20 in distilled water and have a pH of 7.0 (FVIII formulation buffer). Said buffer is used in the experiments described herein if not otherwise stated. Formulations of FVIII may be sterile, e.g., sterile filtered, in particular for in vivo use.

The pharmaceutical composition may be formulated as desired appropriate by the skilled person, e.g., for intravenous (i.v.) or subcutaneous application, intraperitoneal or intramuscular application. Generally, it is for administration as slow i.v. push bolus injection. Continuous infusion is indicated e.g., for patients requiring admission for severe bleeds or surgical procedures. Oral application, which may contribute to tolerance induction, is also possible, e.g., after expression in plants. The pharmaceutical composition may be for slow release.

Pharmaceutical compositions comprising FVIII can be lyophilized. Dosages and treatment schemes may be chosen as appropriate, e.g., for prophylaxis of bleeding or with intermittent, on-demand therapy for bleeding events. Decisions on dosing may be made by the physician. Dosing depends on the patent, e.g., weight, FVIII status etc. For example, the FVIII of the invention may be administered in dosages of 0.5 to 250 IU/kg body weight every 0.5 to 6 days intravenously depending on the severity of the disease, typically, 0.5 to 200 IU/kg body weight. The invention also provides a pharmaceutical composition comprising the FVIII protein of the invention in combination with an immunosuppressive agent (e.g., methylprednisolone, prednisolone, dexamethasone, cyclophosphamide, rituximab, and/or cyclosporin), and/or it may be for administration at substantially the same time with (e.g. within minutes to 12 hours) with such an agent.

The pharmaceutical composition, e.g., comprising the protein of the invention, may be for use in treating a patient in need thereof, in particular, a Hemophilia A patient, e.g., a patient with acquired hemophilia involving an autoimmune response to FVIII or a congenital Hemophilia A patient. Mammals such as mice or dogs may be treated with the pharmaceutical composition of the invention, but the patient typically is a human patient.

The pharmaceutical composition of the invention may also be used for treatment of a patient previously treated with a recombinant and/or plasmatic Factor VIII protein. In a patient who has an antibody including an inhibitory antibody response to a recombinant and/or plasmatic Factor VIII protein, the pharmaceutical compositions may, e.g., be used for immune tolerance induction (ITI) treatment. The compositions of the invention may thus also be used for rescue ITI. The pharmaceutical compositions may also be advantageously used in a patient who has had an antibody response including an inhibitory antibody response to a recombinant and/or plasmatic Factor VIII protein, e.g., who has been treated by ITI. The pharmaceutical compositions may also be advantageously used in a patient who has had an antibody response including an inhibitory antibody response to a recombinant and/or plasmatic Factor VIII protein, who has not been treated by ITI.

The invention also provides a vial comprising the pharmaceutical composition of the invention, e.g., a syringe. The syringe may be a pre-filled syringe, e.g., a ready-to-use syringe.

All publications cited herein are fully incorporated herewith. The invention is further illustrated by the following examples, which are not to be understood as limiting the scope of the invention.

### EXAMPLES

### 1. Generation of Single Chain Variants and Determination of Biological Activity

During the development of a new recombinant hemophilia A therapeutic, the possibility of using a single chain FVIII molecule lacking the furin cleavage recognition site and thereby inhibiting enzymatic cleavage has been tested. Said single chain FVIII molecules may be favorable in terms of stability during purification and storage, but may also have benefits in vivo in terms of stability e.g. for subcutaneous administration and plasma half-life. Therefore, DNA plasmids encoding different B-domain truncated FVIII single chain variants each having individual deletions including the furin cleavage recognition site were designed and their functionality assessed using in vitro assays.

### Material and Methods

### Preparation of Constructs

As a basic FVIII sequence for cloning, a codon-optimized sequence of Refacto AF® was used, wherein for simplifying cloning, 6 restriction sites were added through silent mutations. Some of these restriction sites were again excluded due to codon-optimization. The basic sequence is AC-6rs-REF (SEQ ID NO: 28).

For the constructs encoding the FVIII of the invention and comparative constructs also analysed in this context, either the complete FVIII sequence or DNA regions encoding 550-600 bp from the FVIII a2 domain to the A3 domain were synthesized. The complete synthesized DNA was codon-optimized. The DNA fragments were 5′ terminally flanked by an EcoRV restrictions site, and 3′ terminally flanked by an EcoRI restriction site, and these restriction sites were also present in the basic FVIII sequence used. Restriction of the DNA inserts and the FVIII backbone plasmid allowed for targeted ligation and generation of FVIII single chain plasmids. Completely synthesized FVIII DNA was 5′ terminally flanked by a HindIII restrictions site, and 3′ terminally flanked by a Irestriction site.

By transformation of E. coli K12 with said plasmids, expansion of transformed bacteria under ampicillin selection and plasmid preparation, large amounts of the plasmids could be prepared. Genetic engineering work was carried out by Thermo Fisher Scientific after design with VectorNTl Software (Thermo Fisher Scientific, Massachusetts, USA).

### Cultivation of CAP-T Cells

CAP-T cells (Cevec Pharmaceuticals, Köln, Germany) were cultured in PEM medium supplemented with 4 mM GlutaMAX (Thermo Fisher Scientific, 35050038) and 5 μg/ml blasticidin (Thermo Fisher Scientific, R21001; complete PEM medium). In order to thaw the cells, the required amount of frozen vials were transferred to a 37° C. water bath. After thawing, each vial was transferred to 10 ml of chilled, complete PEM medium. The cell suspension was centrifuged at 150×g for 5 minutes. During this washing step the dimethyl sulfoxide (DMSO) used for cryopreservation was removed. The pellet was resuspended in 15 ml warm, complete PEM medium and transferred to a 125 ml shaker flask. The cells were incubated at 37° C. in a humidified incubator with an atmosphere containing 5% CO₂. The flasks were set on a shaking platform, rotating at 185 rpm with an orbit of 50 mm.

Subculturing of the cells was performed every 3 to 4 days. The fresh culture was set to 0.5×10⁶ cells/ml by transferring the required amount of cultured cell suspension to a new flask and adding complete PEM medium. In the case that the transferred cell suspension would exceed 20% of the total volume, the suspension was centrifuged at 150×g for 5 minutes and the pellet was resuspended in fresh complete PEM medium. The volume of cell suspension per shaking flask was 20% of the total flask volume.

A minimum of three subcultures were performed after thawing before transfection experiments were performed.

### Protein Expression in CAP-T Cells by Transient Transfection

The CAP-T cells were transfected using the 4D-Nucleofector™ (Lonza, Basel, Switzerland). For each transfection 10×10⁶ CAP-T cells were centrifuged at 150×g for 5 minutes in 15 ml conical tubes. The cells were resuspended in 95 μl supplemented SE Buffer, taking into account the volume of the pellet and the volume of the plasmid solution. Afterwards, 5 μg of the respective plasmid were added to the cell suspension followed by gentle mixing. The solution was transferred to 100 μl Nucleocuvettes. The used transfection program was ED-100. After the transfection, the cells from one Nucleocuvette were transferred to 125 ml shaker flasks, containing 12.5 ml complete PEM medium. The cells were cultivated for 4 days as described above. At day 4 the cells were harvested by centrifugation at 150×g for 5 minutes. Larger protein amounts could be produced by combining 12.5 ml approaches as described above.

### Chromogenic FVIII Activity

The activity of FVIII was determined by a chromogenic assay. In this two-step assay, FIXa and FVllla activate FX in the first step. In the second step, the activated FX hydrolyses a chromogenic substrate, resulting in a color change, which can be measured at 405 nm. Due to the fact that calcium and phospholipids are present in optimal amounts and an excess of FIXa and FX is available, the activation rate of FX is only dependent on the amount of active FVIII in the sample.

The reagents for this chromogenic FVIII activity assay were taken from the Coatest® SP FVIII Kit. The kit contained phospholipids, calcium chloride (CaCl₂), trace amounts of thrombin, the substrate S-2765, a mixture of FIXa and FX and the thrombin inhibitor I-2581. The inhibitor was added, in order to prevent hydrolysis of the substrate by thrombin, which was built during the reaction. All dilutions were performed in distilled water or Tris-BSA (TBSA) Buffer, containing 25 mM Tris, 150 mM sodium chloride (NaCl) and 1% Bovine serum albumin (BSA), set to pH 7.4. Each sample was diluted at least 1:2 with FVIII-depleted plasma. Further dilutions were performed using the TBSA Buffer.

The assay was performed using the BCS XP (Siemens Healthcare, Erlangen, Germany), a fully automated hemostasis analyzer. All reagents including water, TBSA Buffer and the samples were inserted into the analyzer. For each sample the analyzer mixed 34 μl calcium chloride, 20 μl TBSA Buffer, 10 μl sample, 40 μl water, 11 μl phospholipids and 56 μl FIXa-FX-mixture. This mixture was incubated for 300 seconds. Afterwards, 50 μl of S-2765+I-2581 were added to the reaction. Upon addition of the substrate, the absorption at 405 nm was measured for 200 seconds.

In order to calculate the amount of active FVIII, the software of the analyzer evaluated the slope of the measured kinetic between 30 seconds and 190 seconds after starting the reaction. This result was correlated to a calibration curve, generated with a biological reference preparation (BRP) of FVIII. The activity of the BRP is indicated in IU/ml. However, IU/ml can be assumed equivalent to U/ml. The results were indicated as “% of normal”. These results were converted to U/ml, as 100% of normal FVIII activity are equivalent to 1 U FVIII activity per ml.

### Clotting Activity FSL

In addition to the two-stage chromogenic assay (see above), a one-stage clotting assay was also performed in order to determine the amount of active FVIII. During this assay, FVIII-depleted plasma, CaCl₂, the activator Actin FSL and the FVIII-containing sample are mixed in one step. The activator leads to the generation of FXIa, which activates FIX. FVIIIa, FIXa and FX built the tenase complex and FX becomes activated. Further activation of prothrombin and fibrinogen finally leads to the formation of a fibrin clot. The time needed to form the clot, the activated partial thromboplastin time (aPTT), is measured. The aPTT varies, depending on the amount of FVIII.

The clotting assay was performed using the BCS XP. TBSA Buffer, FVIII-depleted plasma, Actin FSL, CaCl₂ and the sample were inserted into the analyzer. The sample was diluted at least 1:2 with FVIII-depleted plasma. Further dilutions were performed using the TBSA Buffer. For each sample the analyzer mixed 45 μl TBSA Buffer, 5 μl sample, 50 μl FVIII-depleted plasma and 50 μl Actin FSL. The reaction was started by the addition of 50 μl CaCl₂. The analyzer measured the time needed for clot formation.

In order to calculate the amount of active FVIII, the software of the analyzer evaluated a baseline extinction at 405 nm at the beginning of the reaction. All of the following extinction values, within a time of 200 seconds, were analysed regarding their difference to the baseline extinction. The first time point exceeding a defined threshold was determined as the clotting time. This result was correlated to a calibration curve, generated with a BRP of FVIII.

### FVIII Antigen ELISA

The amount of FVIII antigen was determined using the Asserachrom® VIII:Ag ELISA (Diagnostica Stago, Asnières sur Seine Cedex, France). In this sandwich ELISA, the applied FVIII is bound by mouse monoclonal anti-human FVIII F(ab′)₂ fragments, which are coated to the plate by the manufacturer. The detection of the bound FVIII occurs via mouse monoclonal anti-human FVIII antibodies, which are coupled to a peroxidase. In the case that FVIII is present, the peroxidase-coupled antibody binds to FVIII and can be detected by the addition of a tetramethylbenzidine (TMB) solution. TMB turns from a clear to a blue-green solution upon reaction with peroxidase. After a short time, this reaction is stopped by the addition of sulfuric acid (H₂SO₄), which turns the solution yellow. The amount of bound FVIII correlates with the intensity of the yellow color, which can be measured at 450 nm. The final amounts of FVIII are calculated using a calibration curve generated by the measurement of at least five serial dilutions of a calibrator with a known antigen concentration.

The supplied calibrator and control were reconstituted with 500 μl of distilled water, 30 minutes before starting the ELISA. After this incubation time, the calibrator was diluted 1:10 in the supplied phosphate buffer. This represented the starting concentration. The calibrator was further serially diluted 1:2 up to a dilution of 1:64. As the concentration of the calibrator contained approximately 1 U/ml FVIII, depending on the batch, the starting concentration was equivalent to 0.1 U/ml FVIII whereas the last dilution contained approximately 0.0016 U/ml FVIII. The control was diluted 1:10 and 1:20 with the phosphate buffer. All samples were diluted with the phosphate buffer, depending on their previously determined activity (see above) with the aim to be in the middle of the calibration curve. After the dilution of FVIII samples, control and calibrator, 200 μl of each solution were applied per well in duplicates. In addition to that, two wells were filled with 200 μl of phosphate buffer as a blank control. The plate was incubated for 2 hours at room temperature covered with a film. During this time, the peroxidase-coupled anti-human FVIII antibodies were reconstituted with 8 ml phosphate buffer and incubated 30 minutes at room temperature. After the antigen immobilization, the wells were washed five times with the supplied washing solution, which was previously diluted 1:20 with distilled water. Immediately after the washing, 200 μl of the peroxidase-coupled anti-human FVIII antibodies were added to each well and incubated for 2 hours at room temperature covered by a film. Afterwards, the plate was washed five times as before. In order to reveal the amount of bound FVIII, 200 μl of TMB solution were added to each well and incubated for exactly 5 minutes at room temperature. This reaction was stopped by the addition of 50 μl 1 M H₂SO₄ to each well. After an incubation time of 15 minutes at room temperature, the absorbance of each well was measured at 450 nm using the POLARstar Omega plate reader (BMG LABTECH, Ortenberg, Germany).

The results of the ELISA were calculated using the MARS software (BMG Labtech). In a first step, all wells were blank corrected and the mean of the duplicates was calculated.

Afterwards, a 4-parameter fit was applied, in order to calculate the concentrations from the calibration curve. According to this calibration curve the amount of FVIII antigen in each well was determined. In the last step, the values were corrected by the dilution factor, resulting in the FVIII antigen amount of each sample.

### Results and Discussion

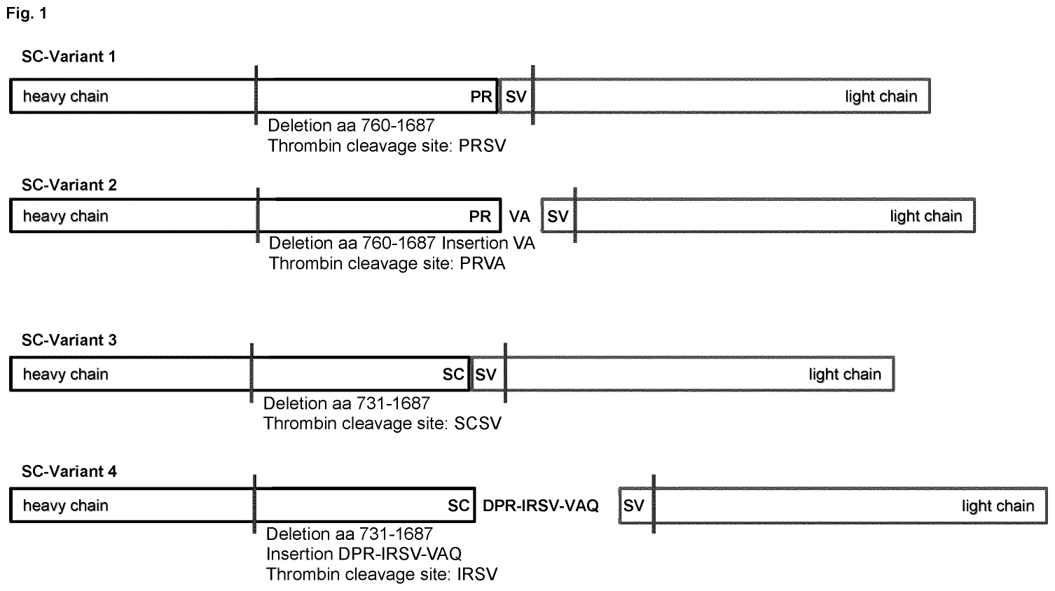

Different variants of single chain FVIII molecules (FIG. 1, 2) were generated and analysed. In all variants, the furin cleavage recognition site was deleted. Variants AC_SC-V1 (V1) and -V3 (V3) have a natural thrombin cleavage site (PR/SV or SC/SV, respectively), and there is only a single deletion of aa 760-1687 (V1) and aa 731-1687 (V3). Variants AC_SC-V2 (V2) and -V4 (V4), compared to V1 and V3, comprise a different thrombin cleavage site (PR/VA or IR/SV, respectively), wherein V2, based on V1, further comprises a VA sequence which can be considered as being derived from the B-domain. V4, based on V3, comprises an insertion DPR-IRSV-VAQ at the site of the deletion (FIG. 1). None of the constructs V1-V4 comprise a processing sequence as required by the present invention, e.g., including the sequence NPP in the context of the thrombin cleavage site of interest.

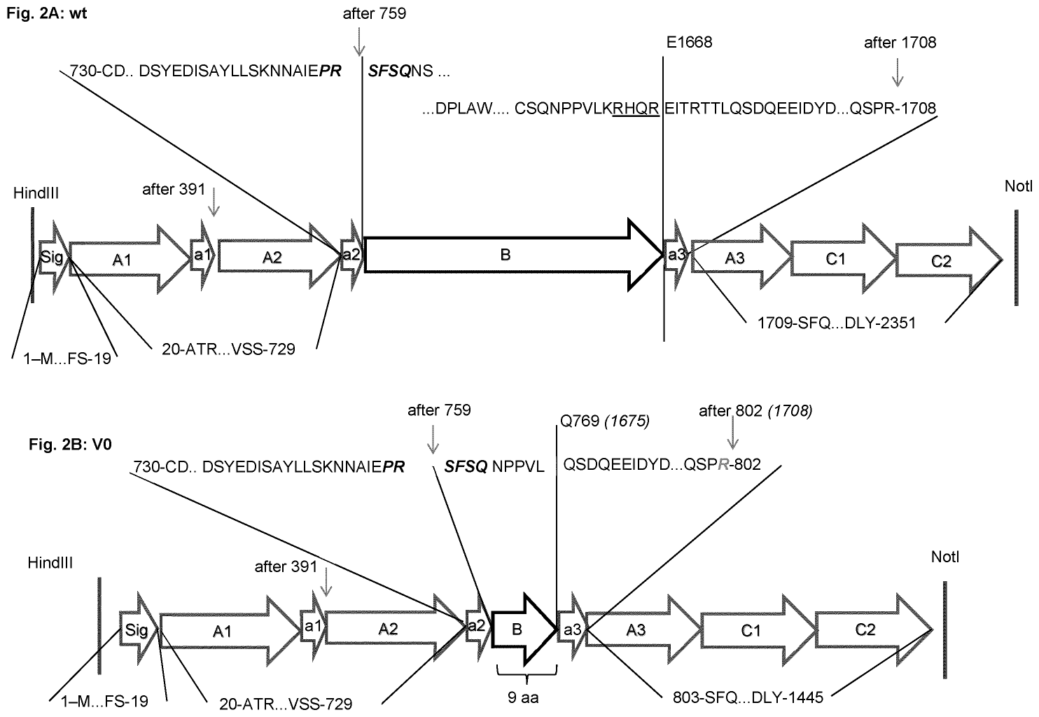

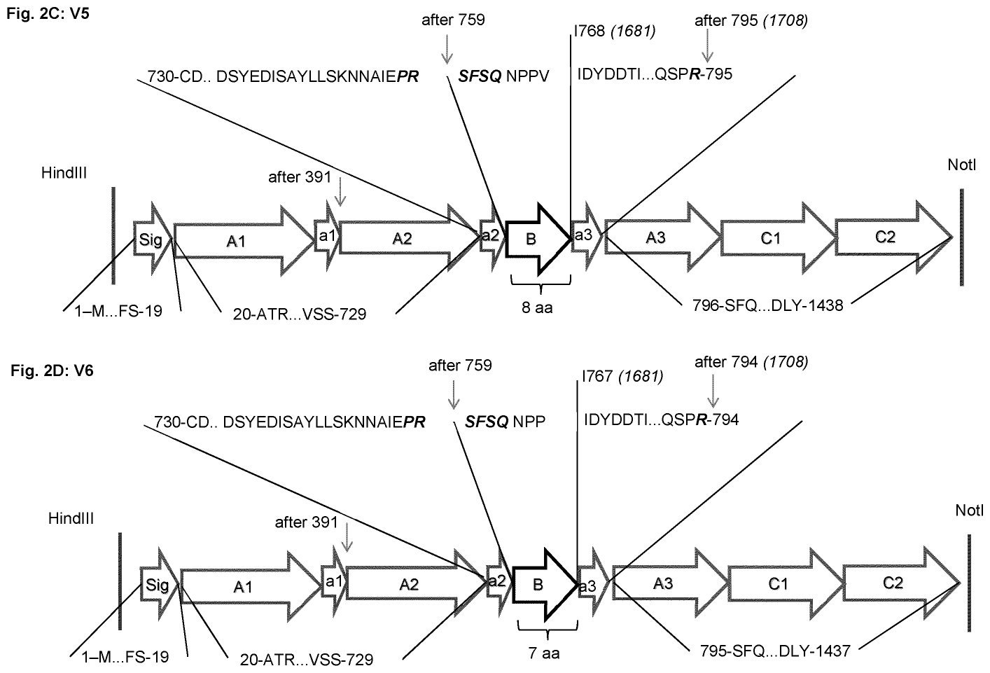

The inventors performed further experimentation by generating additional constructs, designated AC_SC-V0, -V5, -V6 and -V7 (or, short, V0, V5, V6 and V7), shown in FIG. 2. In a less straightforward manner, they moved from a single deletion of the B-domain to creating two deletions, which are bridged by retaining a short fragment of the B-domain. Thus, a processing sequence was generated, which seemed to be closer to the wild type sequence. The constructs are based on the Refacto AF® amino acid sequence AC-6rs-Ref, by introduction of two deletions, i.e. proteins of the invention comprising a processing sequence as defined herein. Thus, V0, V5, V6 and V7 represent FVIII proteins according to the present invention.

Western-Blots of V0-V7 show expression of all analysed constructs mainly as a single chain molecule (not shown), which is indicated by a double band at about 180 kD, and absence or low presence of a heavy chain band at about 80 kD.

The expression levels and in vitro functionality of different single chain FVIII variants were evaluated in comparison to the double chain FVIII variant, AC-6rs-REF, having the identical amino acid sequence as ReFacto AF® and used as baseline molecule during the hemophilia A therapeutic development. Naturally, this double-chain FVIII should be expected to deliver the best functional results.

As described above, CAP-T cells were transiently transfected in duplicate with respective plasmid DNA encoding for either AC-6rs-REF or the different single chain molecules. After four days of cultivation, cells were centrifuged and cell culture supernatants were directly used for determining (I) the chromogenic FVIII activity, (II) the FVIII antigen corresponding to the total FVIII protein amount, and (III) the FVIII clotting activity induced via Actin FSL.

In a comparison of the constructs V1-V4 with the FVIII double-chain molecule AC-6rs (SEQ ID NO: 29), the chromogenic activity and specific chromogenic activity, demonstrating the ratio of chromogenic activity and FVIII antigen, of the expressed single chain constructs were lower than those of the double chain molecule. In more detail, the chromogenic activity and specific chromogenic activity of V3 and V4 were lower than that of V1 and V2. In turn, the chromogenic activity and specific chromogenic activity of the expressed constructs V1 and V2 was still lower than that of the double-chain molecule (data not shown).

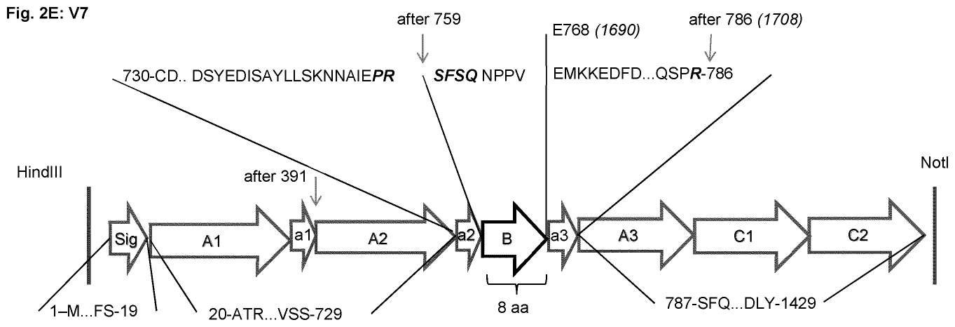

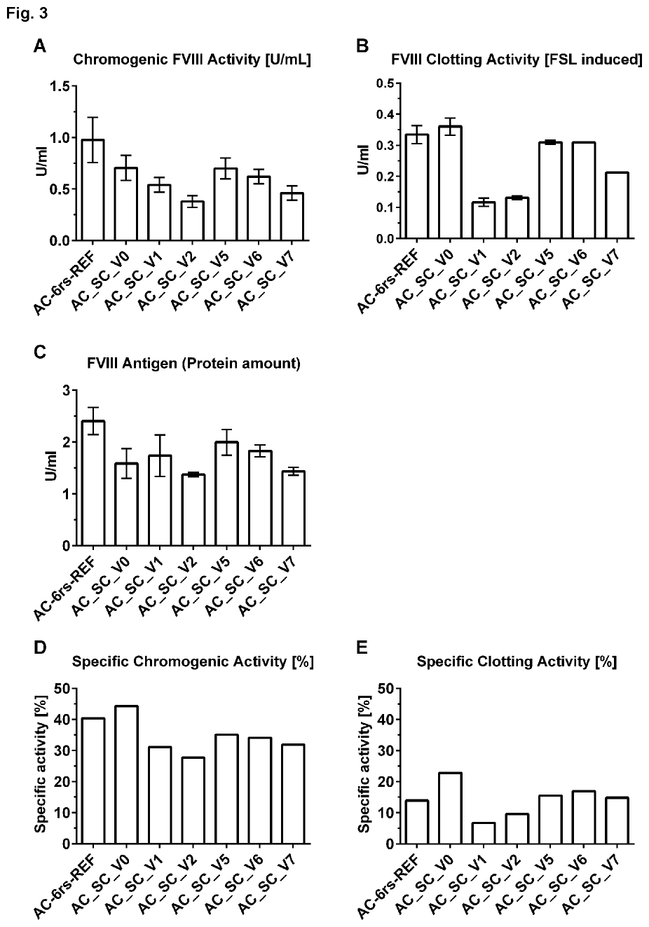

In a comparison of V1, V2, V0, V5, V6, V7 and the double chain FVIII (AC-6rs-REF), as demonstrated in FIG. 3A, the double chain FVIII control reached the highest chromogenic FVIII activity levels of approx. 1 U/ml (all determined in supernatants from transfected cells). Single chain variants AC_SC-V0, -V5 and -V6 demonstrated chromogenic activities of approx. 0.7 U/ml while AC_SC-V1, -V2 and -V7 exhibited approx. 0.4 to 0.5 U/ml.

FVIII protein amounts were also found to be highest in the double chain AC-6rs-REF control with approx. 2.4 U/ml (FIG. 3C). Single chain variants were expressed in the range of 1.4 U/ml for AC_SC-V2 up to 2.0 U/ml for AC_SC-V5. Compared to the two stage chromogenic FVIII assay, the one stage clotting assay generally resulted in lower activity values (FIG. 3B). AC_SC-V0, AC-6rs-REF, AC_SC-V5, and AC_SC-V6 revealed approx. 0.3 U/ml while AC_SC-V7 demonstrated clotting activities of about 0.2 U/ml and AC_SC-V1 and -V2 demonstrated very low clotting activities.

Specific chromogenic activities represent the ratio of chromogenic FVIII activity and FVIII Antigen levels, i.e., they represent the decisive measure of activity. From the patient's view, a high specific activity is desirable, because it means that one can achieve better treatment with less material. In this context, the specific activity was calculated as chromogenic FVIII activity to FVIII antigen ratio displayed in %. As shown in FIG. 3D, the highest specific activity was observed for AC_SC-V0 (44%). The double chain AC-6rs-REF control reached a specific activity of 40%, followed by activities of AC_SC-V5,-V6 and -V7. The specific clotting activity was calculated as FVIII clotting activity to FVIII antigen ratio displayed in %. As shown in FIG. 3E, the highest specific activity was observed for AC_SC-V0 (22.8%) followed by -V6 (16.9%), -V5 (15.5%), -V7 (14.8%).The double chain AC-6rs-REF control reached a lower specific clotting activity of 13.9%, while the single chain constructs AC_SC-V1 and -V2 (prior art) reached only 6.7% and 9.6%, respectively.

In conclusion, the different single chain FVIII molecules AC_SC-V0, -V1, -V2, -V5, -V6, and -V7 were assessed for their in vitro functionality in comparison to the double chain FVIII, AC-6rs-REF. Therefore, all FVIII molecules were analyzed using a two stage chromogenic activity assay, a one stage clotting assay and a FVIII antigen ELISA detecting the total FVIII protein amount. It was observed that all FVIII variants were expressed, since FVIII antigen levels could be determined. In addition, all molecules were generally functional as activity values could be determined in both assays, chromogenic and clotting. However, lowest activities were determined for the single chain variants AC_SC-V1 and AC_SC-V2. While AC_SC-V7 performed moderate in those activity assays, AC_SC-V0, -V5, -V6, and -V7 performed best as single chain variants. The double chain AC-6rs-REF control overall presented the highest protein amount and chromogenic FVIII activity. The specific activities representing either the ratio of chromogenic activity to FVIII antigen or the ratio of clotting activity to FVIII antigen, both serving as indicator of protein functionality, were best for AC_SC-V0, AC_SC-V5, AC_SC-V6, and AC_SC-V7.

### 2. Stability and Pharmacokinetic Data

Prior to in vitro stability experiments and in vivo experiments, the FVIII single chain proteins were purified by standard purification procedures comprising cell removal and concentration of the supernatant, subsequent purification of the FVIII proteins via affinity chromatography, and re-buffering into FVIII formulation buffer.

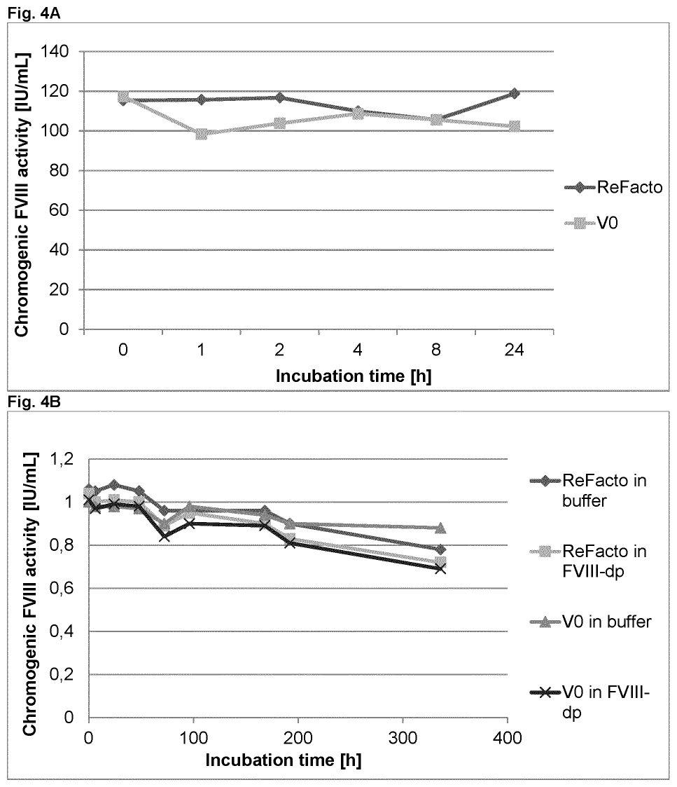

Chromogenic and clotting activity of V0 and ReFacto AF® were analysed over 24 h and 14 days both in buffer and plasma lacking FVIII. V0 and ReFacto AF® were comparable with regard to stability (FIG. 4).

Pharmacokinetic data for V0 and the commercially available double chain FVIII ReFacto AF® were analysed in mice. Coagulation factors (200 IU (chromogenic activity)/kg body weight) were administered in 6 mL/kg body weight by a single intravenous tail vein injection into female haemophilia A mice (FVIII deficient mice, Jax No B6; 129S-F8^{tm1Kaz}/J, Charles River Laboratories, Sulzfeld, Germany). Blood sampling was performed 0.5, 4, 8, 12 and 20 h post treatment and citrate plasma was subsequently extracted by centrifugation. Plasma samples were analysed for the chromogenic FVIII activity and FVIII protein amount (FVIII antigen). Pharmacokinetic evaluation was performed by analysing the raw values and performing a non-compartmental analysis (NCA) using Phoenix WinNonlin 8.1 (Certara, USA).

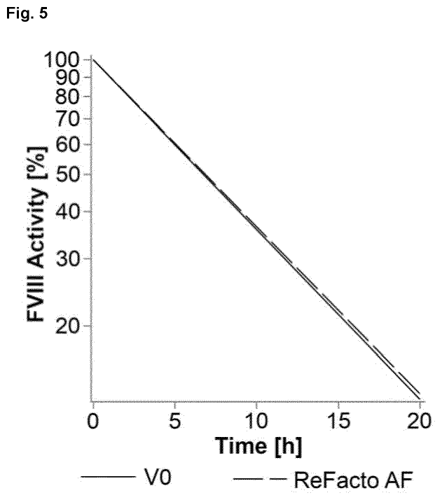

During this study, ReFacto AF® attained a better-than-average half-life of approx. 7.4 h for antigen and 6.9 h for FVIII chromogenic activity. In comparison, V0 showed a half-life of 6.1 and 6.8 h for antigen and chromogenic activity, respectively. Maximal concentrations after intravenous administration were observed for both V0 and ReFacto AF® in the samples taken 0.5 h post injection with values of 2.16 IU/ml and 2.02 IU/ml, respectively. Thus, both variants show a comparable half-life both in relation to antigen and to chromogenic activity (FIG. 5) and comparable maximal concentrations after injection.

Thus, translating the in vitro findings to the animal model, the inventors were able to confirm the advantageous properties of the FVIII variants according to the invention also in the in vivo situation.

1. A recombinant Factor VIII protein comprising, in a single chain, a heavy chain portion comprising an A1 and an A2 domain and a light chain portion comprising an A3, C1 and C2 domain of Factor VIII, wherein,
a) in said recombinant Factor VIII protein, 894 amino acids corresponding to consecutive amino acids between F761 and P1659 of wild type Factor VIII as defined in SEQ ID NO: 1 are deleted, leading to a first deletion;
b) said recombinant Factor VIII protein comprises, spanning the site of the first deletion, a processing sequence comprising SEQ ID NO: 2 or a sequence having at most one amino acid substitution in SEQ ID NO: 2, wherein said processing sequence comprises a first thrombin cleavage site;
c) in said recombinant Factor VIII protein, at least the amino acids corresponding to amino acids R1664 to R1667 of wild type Factor VIII are deleted, leading to a second deletion; and
d) said recombinant Factor VIII protein comprises, C-terminal to the second deletion and N-terminal of the A3 domain, a second thrombin cleavage site.

2. A recombinant Factor VIII protein comprising, in a single chain, a heavy chain portion comprising an A1 and an A2 domain and a light chain portion comprising an A3, C1 and C2 domain of Factor VIII, wherein,
a) said recombinant Factor VIII protein comprises a processing sequence comprising SEQ ID NO: 2 or a sequence having at most one amino acid substitution in SEQ ID NO: 2, wherein said processing sequence comprises a first thrombin cleavage site;
b) directly C-terminal to said processing sequence, said Factor VIII protein comprises a heterologous sequence;
c) directly C-terminal to said processing sequence, or, if present, directly C-terminal to said heterologous sequence, said Factor VIII protein comprises a merging sequence having at least 90% sequence identity to a sequence selected from the group consisting of SEQ ID NO: 9, SEQ ID NO: 10 and SEQ ID NO: 11; and
d) said recombinant Factor VIII protein comprises, C-terminal to SEQ ID NO: 9-11, a second thrombin cleavage site.

3. The recombinant Factor VIII protein of claim 1, wherein the processing sequence is SEQ ID NO: 4 or a sequence having at most one amino acid substitution in said sequence.

4. The recombinant Factor VIII protein of claim 1, wherein the processing sequence is selected from the group consisting of SEQ ID NO: 4, 6, 7 or 8.

5. The recombinant Factor VIII protein of claim 1, wherein the processing sequence is SEQ ID NO: 4.

6. The recombinant Factor VIII protein of claim 1, wherein the amino acids corresponding to amino acids K1663 to L1674 of wild type Factor VIII are deleted, leading to a second deletion.

7. The recombinant Factor VIII protein of claim 1, not comprising a furin cleavage recognition site, and not comprising a sequence having more than 75% sequence identity to the furin cleavage recognition site RHQR between the processing sequence and the merging sequence, preferably, not comprising a sequence having more than 40% sequence identity to SEQ ID NO: 15.

8. The recombinant Factor VIII protein of claim 1, comprising a processing sequence and, C-terminal to said processing sequence, a merging sequence, wherein said sequences are selected from the group consisting of
a. the processing sequence of SEQ ID NO: 4 and a merging sequence of SEQ ID NO: 12,
b. the processing sequence of SEQ ID NO: 5 and a merging sequence of SEQ ID NO: 12,
c. the processing sequence of SEQ ID NO: 6 and a merging sequence of SEQ ID NO: 12,
d. the processing sequence of SEQ ID NO: 7 and a merging sequence of SEQ ID NO: 12,
e. the processing sequence of SEQ ID NO: 8 and a merging sequence of SEQ ID NO: 12,
f. the processing sequence of SEQ ID NO: 3 and a merging sequence of SEQ ID NO: 13,
g. the processing sequence of SEQ ID NO: 2 and a merging sequence of SEQ ID NO: 13,
h. the processing sequence of SEQ ID NO: 3 and a merging sequence of SEQ ID NO: 14.

9. The recombinant Factor VIII protein of claim 1, further comprising a third thrombin cleavage site between the A1 and A2 domain.

10. The recombinant Factor VIII protein of claim 1, that is a fusion protein with at least one heterologous fusion partner selected from the group consisting of an Fc region, an albumin-binding sequence, albumin, PAS polypeptides, HAP polypeptides, the C-terminal peptide of the beta subunit of chorionic gonadotropin, albumin-binding small molecules, polyethylenglycol, hydroxyethyl starch.

11. A nucleic acid encoding a recombinant Factor VIII protein of claim 1, wherein said polynucleotide is an expression vector suitable for expression of said recombinant Factor VIII protein in a mammalian cell.

12. A host cell comprising the nucleic acid of claim 11.

13. A method for preparing a Factor VIII protein, comprising culturing the host cell of claim 12 under conditions suitable for expression of the Factor VIII protein and isolating the Factor VIII protein.

14. A pharmaceutical composition comprising the recombinant Factor VIII protein of claim 1.

15. A method for treating hemophilia A in a subject, comprising administering to the subject an effective amount of the pharmaceutical composition of claim 14.

16. The recombinant Factor VIII protein of claim 2, wherein said Factor VIII protein is a Factor VIII protein of claim 1.

17. The recombinant Factor VIII protein of claim 3, wherein the F, the S C-terminal to the F, the Q or the N are substituted.

18. The recombinant Factor VIII protein of claim 8, wherein said merging sequence is directly C-terminal to said processing sequence.

19. The recombinant Factor VIII protein of claim 10, wherein said heterologous fusion partner is inserted directly C-terminal to said processing sequence.


SEQUENCE LISTING (38 sequences)

SEQ ID NO: 1 - protein, 2351 aa, Homo sapiens
  SIGNAL 1..19: signal
  wt human FVIII
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 9 aa, Artificial Sequence
  minimal processing sequence
PRSFSQNPP

SEQ ID NO: 3 - protein, 10 aa, Artificial Sequence
  Processing sequence
PRSFSQNPPV

SEQ ID NO: 4 - protein, 11 aa, Artificial Sequence
  Processing sequence
PRSFSQNPPVL

SEQ ID NO: 5 - protein, 11 aa, Artificial Sequence
  Processing sequence
  VARIANT 4: Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSXSQNPPVL

SEQ ID NO: 6 - protein, 11 aa, Artificial Sequence
  Processing sequence
  VARIANT 5: Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSFXQNPPVL

SEQ ID NO: 7 - protein, 11 aa, Artificial Sequence
  Processing sequence
  VARIANT 6: Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSFSXNPPVL

SEQ ID NO: 8 - protein, 11 aa, Artificial Sequence
  Processing sequence
  VARIANT 7: Xaa can be any amino acid selected from the group consisting of Ala, Cys, Asp, Glu, Phe, Gly, His, Ile, Lys, Leu, Met, Asn, Pro, Gln, Arg, Ser, Thr, Val, Trp, or Tyr.
PRSFSQXPPVL

SEQ ID NO: 9 - protein, 10 aa, Artificial Sequence
  Merging sequence, e.g., in V0
QSDQEEIDYD

SEQ ID NO: 10 - protein, 7 aa, Artificial Sequence
  merging sequence, e.g., in V5 + V6
IDYDDTI

SEQ ID NO: 11 - protein, 8 aa, Artificial Sequence
  merging sequence, e.g., in V7
EMKKEDFD

SEQ ID NO: 12 - protein, 34 aa, Artificial Sequence
  merging sequence, e.g., in V0
QSDQEEIDYDDTISVEMKKEDFDIYDEDENQSPR

SEQ ID NO: 13 - protein, 28 aa, Artificial Sequence
  merging sequence, e.g., in V6
IDYDDTISVEMKKEDFDIYDEDENQSPR

SEQ ID NO: 14 - protein, 19 aa, Artificial Sequence
  merging sequence, e.g., in V7
EMKKEDFDIYDEDENQSPR

SEQ ID NO: 15 - protein, 11 aa, Homo sapiens
  FVIII sequence comprising furin cleavage recognition site deleted in proteins of the invention
KRHQREITRTT

SEQ ID NO: 16 - protein, 1445 aa, Artificial Sequence
  SIGNAL 1..19
  V0 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 17 - protein, 1423 aa, Artificial Sequence
  SIGNAL 1..19
  V1 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 18 - protein, 1425 aa, Artificial Sequence
  SIGNAL 1..19
  V2 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRVASVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - protein, 1394 aa, Artificial Sequence
  SIGNAL 1..19
  V3 aa incl. signal
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCSVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 20 - protein, 1404 aa, Artificial Sequence
  SIGNAL 1..19
  v4 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDPRIRSVVAQSVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 21 - protein, 1438 aa, Artificial Sequence
  SIGNAL 1..19
  V5 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 22 - protein, 1437 aa, Artificial Sequence
  SIGNAL 1..19
  V6 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 23 - protein, 1429 aa, Artificial Sequence
  SIGNAL 1..19
  V7 aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 24 - DNA, 4335 bp, Artificial Sequence
  V0 na incl. signal sequence without stop codon
  sig_peptide 1..57
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagccccggtccttcagccagaacccccccgtgctgcagagcgaccaggaagagatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgaaacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtac

SEQ ID NO: 25 - DNA, 4314 bp, Artificial Sequence
  V5 na incl. signal sequence without stop codon
  sig_peptide 1..57
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagccccggtccttcagccagaacccccccgtgatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgagacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtac

SEQ ID NO: 26 - DNA, 4311 bp, Artificial Sequence
  V6 na incl. signal sequence withot stop codon
  sig_peptide 1..57
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagccccggtccttcagccagaacccccccatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgagacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtac

SEQ ID NO: 27 - DNA, 4287 bp, Artificial Sequence
  V7 na incl. signal sequence without stop codon
  sig_peptide 1..57
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagccccggtccttcagccagaacccccccgtggagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgagacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtac

SEQ ID NO: 28 - protein, 1457 aa, Artificial Sequence
  SIGNAL 1..19
  SIGNAL 1..19
  AC-6rs-REF aa incl. signal
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 29 - protein, 1533 aa, Artificial Sequence
  SIGNAL 1..19
  29 AC-6rs aa incl. signal sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 30 - DNA, 4371 bp, Artificial Sequence
  AC-6rs-REF na incl. signal sequence without stop codon
  sig_peptide 1..57
atgcagatcgagctgtctacctgcttcttcctgtgcctgctgcggttctgcttcagcgccacccggcggtactacctgggcgccgtggaactgagctgggactacatgcagagcgacctgggcgagctgcccgtggacgccagattccccccaagagtgcccaagagcttccccttcaacacctccgtggtgtacaagaaaaccctgttcgtcgagttcaccgaccacctgttcaatatcgccaagcccagacccccctggatgggcctgctgggccctacaatccaggccgaggtgtacgacaccgtggtcatcaccctgaagaacatggccagccaccccgtgtccctgcacgccgtgggcgtgtcctactggaaggccagcgagggcgccgagtacgacgaccagaccagccagcgcgagaaagaggacgacaaagtctttcctggcggcagccatacctacgtgtggcaggtcctgaaagaaaacggccccatggcctccgaccccctgtgcctgacctacagctacctgagccacgtggacctggtcaaggacctgaacagcggcctgatcggcgccctgctcgtgtgcagagagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcctaagatgcacaccgtgaacggctacgtgaacagaagcctgcccggactgatcggctgccaccggaagtccgtgtactggcacgtgatcggcatgggtaccacccccgaggtgcacagcatctttctggaaggacacaccttcctcgtgcggaaccaccggcaggccagcctggaaatcagccctatcaccttcctgaccgcccagacactgctgatggacctgggccagttcctgctgttttgccacatcagcagccaccagcacgacggcatggaagcctacgtgaaggtggacagctgccccgaggaaccccagctgcggatgaagaacaacgaggaagccgaggactacgacgacgacctgaccgacagcgagatggacgtcgtcagattcgatgacgacaacagccccagcttcatccagatcagaagcgtggccaagaagcaccccaagacctgggtgcactatatcgccgccgaggaagaggactgggactacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccccctgctgtacggcgaagtgggcgacaccctgctgattatcttcaagaaccaggccagccggccctacaacatctacccccacggcatcaccgacgtgcggcccctgtacagcagacggctgcccaagggcgtgaagcacctgaaggacttccccatcctgcccggggagatcttcaagtacaagtggaccgtgaccgtggaagatggccccaccaagagcgaccccagatgcctgacccggtactacagcagcttcgtgaacatggaacgggacctggcctccgggctgatcggccctctgctgatctgctacaaagaaagcgtggaccagcggggcaaccagatcatgagcgacaagcggaacgtgatcctgttcagcgtgttcgatgagaacagaagctggtatctgaccgagaatatccagcggttcctgcccaaccctgccggcgtgcagctggaagatcccgagttccaggccagcaacatcatgcactccatcaatggctacgtgttcgacagcctgcagctgagcgtgtgcctgcacgaggtggcctactggtacatcctgagcatcggcgcccagaccgacttcctgagcgtgttcttcagcggctacaccttcaagcacaagatggtgtacgaggataccctgaccctgttccccttctccggcgaaaccgtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgagcaagaacaacgccatcgagcctcggagcttctcccagaacccccccgtgctgaagcggcaccagagagagatcacccggaccaccctgcagagcgaccaggaagagatcgactacgacgacaccatcagcgtcgagatgaagaaagaggacttcgacatctacgacgaggacgagaaccagagcccccggtccttccagaagaaaacccggcactactttatcgccgccgtggaacggctgtgggactacggcatgagcagcagcccccacgtgctgcggaatagagcccagagcggcagcgtgccccagttcaagaaagtggtgttccaggagttcaccgacggcagcttcacccagcctctgtaccggggcgagctgaacgagcacctgggcctgctgggcccctacatccgggccgaggtggaagataacatcatggtcaccttccggaaccaggccagccggccctacagcttctacagcagcctgatctcctacgaagaggaccagcggcagggcgccgagccccggaagaacttcgtgaagcccaacgagacaaagacctacttctggaaggtgcagcaccacatggcccccaccaaggacgaattcgactgcaaggcctgggcctacttcagcgacgtggacctggaaaaggacgtgcacagcggcctgatcggccccctgctcgtgtgccacaccaacaccctgaaccccgcccacggccggcaggtcacagtgcaggaatttgccctgttcttcaccatcttcgacgagactaagagctggtacttcaccgagaacatggaacggaactgcagagccccctgcaacatccagatggaagatcccaccttcaaagagaactaccggttccacgccatcaatggctacatcatggacaccctgcccggcctggtcatggcccaggaccagagaatccggtggtatctgctgagcatgggcagcaacgagaacatccacagcatccacttcagcggccacgtgttcaccgtgcggaagaaagaagagtacaagatggccctgtacaacctgtaccccggcgtgttcgagacagtggaaatgctgcccagcaaggccggcatctggcgggtggaatgtctgatcggcgagcatctgcacgccggcatgtccaccctgtttctggtgtacagcaacaagtgccagacccccctgggcatggccagcggccacatccgggatttccagatcaccgcctccggccagtacggccagtgggcccctaaactggcccggctgcactacagcggcagcatcaacgcctggtccaccaaagagcccttcagctggatcaaggtggacctgctggcccccatgattatccacggcatcaagacacagggcgccagacagaagttcagcagcctgtacatcagccagttcatcatcatgtacagcctggatggcaagaagtggcagacctaccggggcaacagcaccggcaccctgatggtgttcttcggcaacgtggacagcagcggcatcaagcacaacatcttcaacccccccatcattgcccggtacatccggctgcaccccacccactacagcatccggtccaccctgcggatggaactgatgggctgcgacctgaacagctgctccatgcctctgggcatggaaagcaaggccatcagcgacgcccagatcacagccagcagctacttcaccaacatgttcgccacctggtccccatccaaggccagactgcatctgcagggcagaagcaatgcctggcggcctcaggtcaacaaccccaaagaatggctccaggtggacttccagaaaaccatgaaggtcacaggcgtgaccacccagggcgtgaagtccctgctgacctctatgtacgtgaaagagttcctgatctccagcagccaggacggccaccagtggaccctgttctttcagaacggcaaagtgaaagtgttccagggcaaccaggactccttcacccccgtggtcaactccctggaccccccactgctgaccagatacctgagaatccacccccagagctgggtgcaccagatcgccctgagaatggaagtgctgggatgcgaggcccaggatctgtac

SEQ ID NO: 31 - protein, 9 aa, Artificial Sequence
  merging sequence in V1
SVEMKKEDF

SEQ ID NO: 32 - protein, 20 aa, Homo sapiens
  sequence N-terminal to processing sequence and including 2 aa of processing sequence, i.e., sequence N-terminal of thrombin cleavage site
DSYEDISAYLLSKNNAIEPR

SEQ ID NO: 33 - protein, 5 aa, Homo sapiens
  sequence from the B domain
DPLAW

SEQ ID NO: 34 - protein, 4 aa, Artificial Sequence
  thrombin cleavage site in V1
PRSV

SEQ ID NO: 35 - protein, 6 aa, Artificial Sequence
  sequence in V2
PRVASV

SEQ ID NO: 36 - protein, 4 aa, Artificial Sequence
  sequence in V3
SCSV

SEQ ID NO: 37 - protein, 14 aa, Artificial Sequence
  sequence in V4
SCDPRIRSVVAQSV

SEQ ID NO: 38 - protein, 10 aa, Artificial Sequence
  insertion in V4
DPRIRSVVAQ